(19) United States
(12) Patent Application Publication
(10) Pub. No.: US 2025/0170133 A1
(43) Pub. Date: May 29, 2025

(54) PHARMACEUTICAL COMBINATIONS AND METHODS OF USE OF AMINO-PYRROLOPYRIMIDINONE COMPOUND

(71) Applicants: Merck Sharp & Dohme LLC, Rahway, NJ (US); ArQule Inc., Wilmington, DE (US); Ohio State Innovation Foundation, Columbus, OH (US)

(72) Inventors: Mohammed Z.H. Farooqui, Gwynedd, PA (US); Patricia Marinello, Philadelphia, PA (US); Anson Kunjachan Abraham, Hopkinton, MA (US); Sudharshan Eathiraj, Shrewsbury, MA (US); Brian E. Schwartz, Boca Raton, FL (US); Yi Yu, North Easton, MA (US); John C. Byrd, Cincinnati, OH (US); Elizabeth M. Muhowski, Rochester, MN (US); Jennifer A. Woyach, Columbus, OH (US)

(73) Assignees: Merck Sharp & Dohme LLC, Rahway, NJ (US); ArQule Inc., Wilmington, DE (US); Ohio State Innovation Foundation, Columbus, OH (US)

(21) Appl. No.: 18/848,613

(22) PCT Filed: Mar. 27, 2023
(86) PCT No.: PCT/US2023/016420

§ 371 Date: Sep. 19, 2024

Related U.S. Application Data
(60) Provisional application No. 63/324,932, filed on Mar. 29, 2022.

(51) Int. Cl. A61K 31/519; A61K 31/635; A61P 35/02

(52) U.S. Cl. CPC A61K 31/519; A61K 31/635; A61P 35/02

(57) ABSTRACT

The application relates to a pharmaceutical combination or combinational therapy comprising a therapeutically effective amount of the compound of Formula (I), or a pharmaceutically acceptable salt thereof, and at least one second agent, or a pharmaceutically acceptable salt thereof, for use in the treatment of a BTK-mediated disease or disorder.

### BACKGROUND OF THE INVENTION

BTK is a member of the Tec family of tyrosine kinases and plays an important role in the regulation of early B-cell development and mature B-cell activation and survival. Functioning downstream of multiple receptors, such as growth factors, B-cell antigen, chemokine, and innate immune receptors, BTK initiates a number of cellular processes including cell proliferation, survival, differentiation, motility, angiogenesis, cytokine production, and antigen presentation.

BTK-deficient mouse models have shown the role BTK plays in allergic disorders and/or autoimmune disease and/or inflammatory disease. For instance, BTK deficiency in standard murine preclinical models of systemic lupus erythematosus (SLE) has been shown to result in a marked amelioration of disease progression. Furthermore, BTK-deficient mice can be resistant to developing collagen-induced arthritis and less susceptible to Staphylococcus-induced arthritis. Due to BTK's role in B-cell activation, BTK inhibitors can also be useful as inhibitors of B-cell mediated pathogenic activity (such as autoantibody production). Expression of BTK in osteoclasts, mast cells and monocytes has been shown to be important for the function of these cells. For example, impaired IgE-mediated mast cell activation and reduced TNF-alpha production by activated monocytes has been associated with BTK deficiency in mice and humans. Thus, BTK inhibition can be useful for the treatment of allergic disorders and/or autoimmune and/or inflammatory diseases such as: SLE, rheumatoid arthritis, multiple vasculitides, idiopathic thrombocytopenic purpura (ITP), myasthenia gravis, allergic rhinitis, multiple sclerosis and asthma.

BTK's role in apoptosis demonstrates the utility of inhibition of BTK activity for the treatment of cancers, B-cell lymphoma, leukemia, and other hematological malignancies. In addition, given the role of BTK in osteoclast function, inhibition of BTK activity can be useful for the treatment of bone disorders such as osteoporosis.

Many antagonists of B-cell receptor (BCR) signaling, especially inhibitors of BTK, have demonstrated clinical efficacy in the treatment of chronic lymphocytic leukemia (CLL). While therapeutically effective, BTK inhibition alone is not sufficient to eliminate disease, as seen with only a small subset of patients treated with the BTK inhibitor (BTKi) ibrutinib reaching complete responses and very rarely achieving undetectable minimal residual disease (uMRD) (Ahn i.e., Farooqui MZH, Tian X, Valdez J, Sun C, Soto S, et al. Depth and durability of response to ibrutinib in CLL: 5-year follow-up of a phase 2 study. Blood. 2018; 131(21):2357-66.). Previous studies show that prolonged BTK inhibition increases dependence on B-Cell Lymphoma 2 (BCL2), an anti-apoptotic protein highly expressed in CLL (Deng J, et al., Leukemia. 2017; 31(10):2075-84). In a phase 1 clinical trial, patients with relapsed/refractory (R/R) CLL treated with the BCL2 inhibitor venetoclax as a monotherapy had an overall response rate of 79%; however, only 5% of patients had uMRD in the bone marrow Roberts A W, et al., N Engl J Med. 2016; 374(4):311-22). Results from a phase 2 clinical trial investigating the combination of ibrutinib and venetoclax as a frontline treatment show that after 18 cycles of combinatorial therapy, 96% of patients had reached complete remission and 69% of patients had uMRD in the bone marrow, demonstrating the clinical efficacy of this combination (Jain N, et al., N Engl J Med. 2019; 380(22):2095-103).

The compound of the instant invention, nemtabrutinib (also known as MK1026 and ARQ 531) is a potent, reversible BTK inhibitor that has demonstrated greater inhibition of BTK's downstream targets compared to ibrutinib, as well as efficacy in R/R disease (Reiff S D, Mantel R, Smith L L, Greene J T, Muhowski E M, Fabian C A, et al. The BTK Inhibitor ARQ 531 Targets Ibrutinib-Resistant CLL and Richter Transformation. Cancer Discovery. 2018; 8(10):1300-15. Combinations of nemtabrutinib with other therapeutic agents, such as BCL2 inhibitors, are of particular interest in providing much needed therapies for treating BTK-mediated disorders/diseases. One such combinatorial therapy is nemtabrutinib with venetoclax for treating patients who have relapsed/refractory CLL/SLL following at least 1 line of prior therapy.

### SUMMARY OF THE INVENTION

A first aspect of the application relates to a pharmaceutical composition comprising a therapeutically effective amount of a compound of Formula (I):

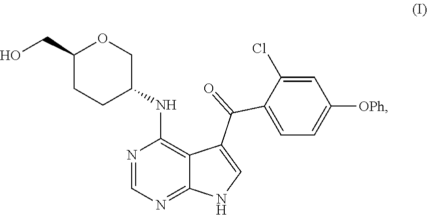 [SMILES: O=C(c1ccc(Oc2ccccc2)cc1Cl)c1c[nH]c2ncnc(N[C@@H]3CC[C@@H](CO)OC3)c12]

or a pharmaceutically acceptable salt thereof, in combination with a therapeutically effective amount of at least one second agent, or a pharmaceutically acceptable salt thereof, for use in the treatment of a BTK-mediated disease or disorder. As used herein, the expressions “compound of Formula (I)”. “Compound (I),” or nemtabrutinib refer to the same compound and can be used interchangeably.

### BRIEF DESCRIPTION OF THE DRAWINGS

FIG. 1A depicts a chart comparing data for cells treated with venetoclax, Compound 1, ibrutinib and combinations.

FIG. 1B depicts a representative immunoblot.

FIGS. 1C-1E depict charts comparing data for cells treated with venetoclax, Compound 1, ibrutinib and combinations, for various proteins (FIG. 1C is pBTK, FIG. 1D is pERK and FIG. 1E is MCL-1).

FIG. 1F depicts a chart showing data for cells that were co-cultured with NK Tert stromal cells performing BH3 profiling using BIM, BAD, MS-1 and Y4ek peptides.

FIG. 2A represent the study design for mice treated with vehicle, venetoclax, nemtabrutinib, ibrutinib, venetoclax and nemtabrutinib, or venetoclax and ibrutinib.

FIG. 2B depicts a chart showing the survival data for mice treated with vehicle, venetoclax, nemtabrutinib, ibrutinib, venetoclax and nemtabrutinib, or venetoclax and ibrutinib.

FIG. 2C depicts a chart showing the probability of survival for treatment with vehicle, venetoclax, nemtabrutinib, ibrutinib, venetoclax and nemtabrutinib, or venetoclax and ibrutinib.

### DETAILED DESCRIPTION OF THE INVENTION

A first aspect of the application relates to a pharmaceutical composition comprising a therapeutically effective amount of a compound of Formula (1):

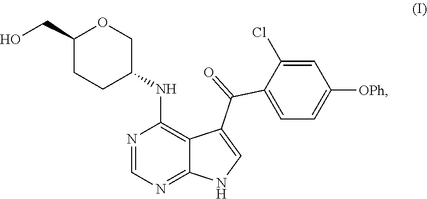 [SMILES: O=C(c1ccc(Oc2ccccc2)cc1Cl)c1c[nH]c2ncnc(N[C@@H]3CC[C@@H](CO)OC3)c12]

or a pharmaceutically acceptable salt thereof, in combination with a therapeutically effective amount of at least one second agent, or a pharmaceutically acceptable salt thereof, for use in the treatment of a BTK-mediated disease or disorder.

Another aspect of the application relates to a method of treating a BTK-mediated disease or disorder in a subject in need, the method comprising co-administering to the subject a therapeutically effective amount of a compound of Formula (I), or a pharmaceutically acceptable salt thereof, in combination with a therapeutically effective amount of at least one second agent, or a pharmaceutically acceptable salt thereof. A further aspect of the invention is the method wherein the compound of Formula (I), or a pharmaceutically acceptable salt thereof, is administered simultaneously or concurrently with a therapeutically effective amount of at least one second agent, or a pharmaceutically acceptable salt thereof. A further aspect of the invention is the method wherein the compound of Formula (I), or a pharmaceutically acceptable salt thereof, is administered daily with a therapeutically effective amount of at least one second agent, or a pharmaceutically acceptable salt thereof.

Another aspect of the application relates to use of a compound of Formula (I), or a pharmaceutically acceptable salt thereof, in combination with at least one second agent, or a pharmaceutically acceptable salt thereof, in the treatment of a BTK-mediated disease or disorder.

Another aspect of the application relates to a compound of Formula (I) or a pharmaceutically acceptable salt thereof for use in a combinational therapy with at least one second agent, or a pharmaceutically acceptable salt thereof, for the treatment of a BTK-mediated disease or disorder.

Another aspect of the application relates to a method of treating cancer, such as hematological cancer, including B-cell lymphoma or non-Hodgkin's lymphoma, wherein the compound of Formula (I), or a pharmaceutically acceptable salt thereof, is administered with a therapeutically effective amount of at least one second agent, or a pharmaceutically acceptable salt thereof.

Another aspect of the application relates to a method of treating cancer, including chronic lymphocytic leukemia (CLL), small lymphocytic lymphoma (SLL) or Mantle cell lymphoma (MCL). Another aspect of the application relates to a method of treating chronic lymphocytic leukemia (CLL) or small lymphocytic lymphoma (SLL).

Another aspect of the application relates to a method of modulating (e.g., inhibiting) or modulation (e.g., inhibition) of BTK by administering a compound of Formula (I), or a pharmaceutically acceptable salt thereof, in combination with at least one second agent, or a pharmaceutically acceptable salt thereof.

Another aspect of the application relates to a pharmaceutical composition comprising a therapeutically effective amount of a compound of Formula (I), or a pharmaceutically acceptable salt thereof, in combination with a therapeutically effective amount of at least one second agent, or a pharmaceutically acceptable salt thereof, for the manufacture of a medicament for the treatment, of a BTK-mediated disease or disorder.

Another aspect of the application relates to use of a compound of Formula (1) or a pharmaceutically acceptable salt thereof in combination with at least one second agent or a pharmaceutically acceptable salt thereof in the manufacture of a medicament for the treatment, of a BTK-mediated disease or disorder.

Another aspect of the application relates to a pharmaceutical composition of a compound of Formula (I), or a pharmaceutically acceptable salt thereof, in combination with at least one second agent, wherein the second agent is selected from Venetoclax, BI-97C1, sabutoclax, navitoclax, obatoclax, 4-[4-[[2-(4-Chlorophenyl)phenyl]methyl]piperazin-1-yl]-N-[4-[[(2R)-4-(dimethylamino)-1-phenylsulfanylbutan-2-yl]amino]-3-nitrophenyl]sulfonylbenzamide (ABT-737), N-[4-(2-tert-butylphenyl)sulfonylphenyl]-2,3,4-trihydroxy-5-[(2-propan-2-ylphenyl)methyl]benzamide (TW-37), APG-1252, APG-2575 or S55746. A further aspect is a pharmaceutical composition of a compound of Formula (I), or a pharmaceutically acceptable salt thereof, in combination with venetoclax or a pharmaceutically acceptable salt thereof.

In another aspect. the pharmaceutical composition of a compound of Formula (I), or a pharmaceutically acceptable salt thereof, in combination with venetoclax comprises from about 30 mg to about 80 mg of the compound of Formula (I) or a pharmaceutically acceptable salt thereof. In a further aspect, the pharmaceutical composition of a compound of Formula (I), or a pharmaceutically acceptable salt thereof, in combination with venetoclax comprises about 45 mg of the compound of Formula (I) or a pharmaceutically acceptable salt thereof. In a further aspect, the pharmaceutical composition of a compound of Formula (I), or a pharmaceutically acceptable salt thereof, in combination with venetoclax comprises about 65 mg of the compound of Formula (I) or a pharmaceutically acceptable salt thereof. In a further aspect, the pharmaceutical composition of a compound of Formula (1), or a pharmaceutically acceptable salt thereof, in combination with venetoclax comprises about 80 mg of the compound of Formula (I) or a pharmaceutically acceptable salt thereof.

In a further aspect, the pharmaceutical composition of a compound of Formula (I), or a pharmaceutically acceptable salt thereof, in combination with venetoclax or a pharmaceutically acceptable salt thereof, comprises from about 30 mg to about 80 mg of the compound of Formula (I) or a pharmaceutically acceptable salt thereof, and about 400 mg of venetoclax or a pharmaceutically acceptable salt thereof. In a further aspect, the pharmaceutical composition of a compound of Formula (I), or a pharmaceutically acceptable salt thereof, in combination with venetoclax comprises about 45 mg of the compound of Formula (I) and about 400 mg of venetoclax. In a further aspect, the pharmaceutical composition of a compound of Formula (1), or a pharmaceutically acceptable salt thereof, in combination with venetoclax or a pharmaceutically acceptable salt thereof, comprises about 65 mg of the compound of Formula (I) and about 400 mg of venetoclax. In a further aspect, the pharmaceutical composition of a compound of Formula (I), or a pharmaceutically acceptable salt thereof, in combination with venetoclax or a pharmaceutically acceptable salt thereof, comprises about 80 mg of the compound of Formula (I) or a pharmaceutically acceptable salt thereof, and about 400 mg of venetoclax or a pharmaceutically acceptable salt thereof.

Another aspect of the application relates to a method of treating a BTK-mediated disorder in a subject in need thereof, where the method comprises co-administering to a subject in need thereof, a therapeutically effective amount of a compound of Formula (I):

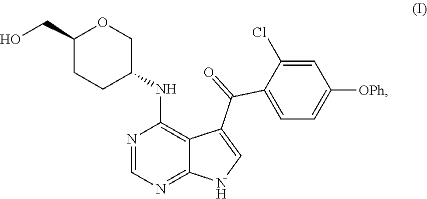 [SMILES: O=C(c1ccc(Oc2ccccc2)cc1Cl)c1c[nH]c2ncnc(N[C@@H]3CC[C@@H](CO)OC3)c12]

or a pharmaceutically acceptable salt thereof, in combination with a therapeutically effective amount of at least one second agent or a pharmaceutically acceptable salt thereof. In a further aspect, the method comprises administering a compound of Formula (I) after administering the second agent. In a further aspect, the BTK-mediated disorder treated is a cancer selected from CLL, SLL or MLL. In a further aspect, the method comprises administering a second agent selected from Venetoclax, BI-97C1, sabutoclax, navitoclax, obatoclax, 4-[4-[[2-(4-Chlorophenyl)phenyl]methyl]piperazin-1-yl]-N-[4-[[(2R)-4-(dimethylamino)-1-phenylsulfanylbutan-2-yl]amino]-3-nitrophenyl]sulfonylbenzamide (ABT-737), N-[4-(2-tert-butylphenyl)sulfonylphenyl]-2,3,4-trihydroxy-5-[(2-propan-2-ylphenyl)methyl]benzamide (TW-37), APG-1252, APG-2575 or S55746. A further aspect is the method where a compound of Formula (I), or a pharmaceutically acceptable salt thereof, is administered with venetoclax or a pharmaceutically acceptable salt thereof. A further aspect is the method where a compound of Formula (I), or a pharmaceutically acceptable salt thereof, is co-administered with venetoclax or a pharmaceutically acceptable salt thereof, wherein the compound of Formula (I), or a pharmaceutically acceptable salt thereof, is administered after administering venetoclax, or a pharmaceutically acceptable salt thereof. In further aspect, the compound of Formula (I) is administered at least 10 hours after venetoclax is administered.

Another aspect of the application relates to a method of treating a BTK-mediated disorder in a subject in need thereof, where the BTK-mediated disorder is a cancer selected from CLL, SLL or MLL, and the method comprises co-administering to a subject in need thereof, a therapeutically effective amount of a compound of Formula (I), or a pharmaceutically acceptable salt thereof, in combination with a therapeutically effective amount of at least one second agent or a pharmaceutically acceptable salt thereof. A further aspect of the application is the method of treating a BTK-mediated disorder, where said second agent is venetoclax or a pharmaceutically acceptable salt thereof. A further aspect of the method comprises administering about 30 mg to about 80 mg of the compound of Formula (I) or a pharmaceutically acceptable salt thereof, after administering about 20 to about 400 mg of venetoclax or a pharmaceutically acceptable salt thereof. In another aspect, the method comprises administering about 45 mg of the compound of Formula (I) or a pharmaceutically acceptable salt thereof, after administering about 400 mg of venetoclax or a pharmaceutically acceptable salt thereof. In another aspect, the method comprises administering about 65 mg of the compound of Formula (I) or a pharmaceutically acceptable salt thereof, after administering about 400 mg of venetoclax or a pharmaceutically acceptable salt thereof. In another aspect, the method comprises administering about 80 mg of the compound of Formula (I) or a pharmaceutically acceptable salt thereof, after administering about 400 mg of venetoclax or a pharmaceutically acceptable salt thereof.

The present application relates to pharmaceutical combinations or combinational therapies that are capable of modulating the activity Bruton's Tyrosine Kinase (BTK). The application features methods of treating a disease or disorder in which BTK plays a role by administering to a patient in need thereof a therapeutically effective amount of the compound of Formula (I), or a pharmaceutically acceptable salt thereof, in combination with a therapeutically effective amount of a second agent, as described herein. The methods of the present application can be used in the treatment of a variety of BTK-mediated diseases and disorders by inhibiting the activity of BTK kinase. Inhibition of BTK provides treatment, prevention, or amelioration of diseases including, but not limited to, immune disorders, cancer, cardiovascular diseases, viral infections, inflammation, metabolism/endocrine function disorders and neurological disorders.

The compound of Formula (I), or pharmaceutically acceptable salts thereof, has a structure:

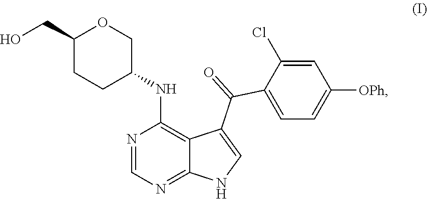 [SMILES: O=C(c1ccc(Oc2ccccc2)cc1Cl)c1c[nH]c2ncnc(N[C@@H]3CC[C@@H](CO)OC3)c12]

also known as nemtabrutinib, MK-1026 and formerly ARQ531, is a reversible non-covalent adenosine triphosphate (ATP) competitive inhibitor of Bruton's tyrosine kinase (BTK) and was designed to target both the wild-type and BTK-C481S mutant. The methods of making this compound are described in U.S. Pat. No. 9,630,968, incorporated herein by reference. The invention encompasses compounds of formula (I) and salts thereof, as described in the '968 patent. Compound (I) (nemtabrutinib) has demonstrated greater inhibition of BTK's downstream targets compared to another BTK inhibitor, ibrutinib. (Reiff S D, Mantel R, Smith L L, Greene J T, Muhowski E M, Fabian C A, et al. The BTK Inhibitor ARQ 531 Targets Ibrutinib-Resistant CLL and Richter Transformation. Cancer Discovery. 2018; 8(10):1300-15.

The present application provides the medical community with a novel pharmacological strategy for the treatment of diseases and disorders associated with BTK kinase. Results from a phase 2 clinical trial investigating the combination of ibrutinib and venetoclax as a frontline treatment showed that after 18 cycles of combinatorial therapy, 96% of patients had reached complete remission and 69% of patients had uMRD in the bone marrow, demonstrating the clinical efficacy of this combination (Jain N, et al., N Engl J Med. 2019; 380(22):2095-103).

The present application provides pharmaceutical combinations or combinational therapies, using the compound of Formula I, or a pharmaceutically acceptable salt thereof, which have an additive or synergistic effects and safety profile relative to known therapies. An aspect of this invention is related to a method of treating a BTK-mediated disease or disorder in a subject in need thereof, the method comprising co-administering to the subject a therapeutically effective amount of a compound of Formula (I), or a pharmaceutically acceptable salt thereof, in combination with a therapeutically effective amount of at least one second agent or a pharmaceutically acceptable salt thereof, wherein the BTK-mediated disease state or disorder is cancer and the second agent is venetoclax or a pharmaceutically acceptable salt thereof.

The details of the application are set forth in the accompanying description below. Although methods and materials similar or equivalent to those described herein can be used in the practice or testing of the present application, illustrative methods and materials are now described. Other features, objects, and advantages of the application will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this application belongs. All patents and publications cited in this specification are incorporated herein by reference in their entireties.

### Definitions

The articles “a” and “an” are used in this application to refer to one or more than one (i.e., at least one) of the grammatical object of the article. By way of example, “an element” means one element or more than one element.

The term “and/or” is used in this application to mean either “and” or “or” unless indicated otherwise.

The application also includes pharmaceutical compositions comprising an effective amount of the compound of Formula (I) and a pharmaceutically acceptable carrier.

The term “carrier”, as used in this application, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The compound of Formula (I) may be in the form of a salt, ester, prodrug, solvate, tautomer, isotope, etc., which are also within the scope of this application and are taught in U.S. Pat. No. 9,630,968, which is incorporated herein by reference.

In the present specification, the structural formula of the compound represents a certain isomer for convenience in some cases, but the present application includes all isomers, such as geometrical isomers, optical isomers based on an asymmetrical carbon, stereoisomers, tautomers, and the like, as shown in U.S. Pat. No. 9,630,968, incorporated herein by reference.

The compound of Formula (I) may be more potent than one or more known BTK inhibitors, including, but not limited to Ibrutinib, GDC-0834, RN486, CGI-560, CGI-1746, HM-71224, CC-292, ONO-4059, CNX-774, LFM-A13, GDC-0853, LOU684, PRN1008, acalabrutinib, zanubrutinib and/or PRN2246, at inhibiting the activity of BTK containing one or more mutations as described herein, e.g., C481S. For example, the compound can be at least about 2-fold, 3-fold, 5-fold, 10-fold, 25-fold, 50-fold or about 100-fold more potent (e.g., as measured by IC50) than Ibrutinib, GDC-0834, RN486, CGI-560, CGI-1746, HM-71224, CC-292, ONO-4059, CNX-774, LFM-A13, GDC-0853, LOU684, PRN1008, acalabrutinib, zanubrutinib and/or PRN2246 at inhibiting the activity of the BTK containing one or more mutations as described herein. A drug-resistant BTK mutant can have without limitation a drug resistance mutation comprising C481S mutation.

The term “administer”, “administering”, or “administration” as used in this application refers to either directly administering a disclosed compound or pharmaceutically acceptable salt of the disclosed compound or a composition to a subject, or administering a prodrug, derivative or analog of the compound or pharmaceutically acceptable salt of the compound or a composition to the subject, which can form an equivalent amount of active compound within the subject's body.

A “patient” or “subject” is a mammal, e.g., a human, mouse, rat, guinea pig, dog, cat, horse, cow, pig, or non-human primate, such as a monkey, chimpanzee, baboon or rhesus.

An “effective amount” or “therapeutically effective amount” when used in connection with a compound or pharmaceutical composition is an amount effective for treating or preventing a disease in a subject as described herein.

The term “treating” with regard to a subject, refers to improving at least one symptom of the subject's disorder. Treating includes curing, improving, or at least partially ameliorating the disorder.

The compound of the present application, or a pharmaceutically acceptable salt thereof, can also be used in combination with a second therapeutic agent to prevent a disease, condition or disorder. As used herein, “preventing” or “prevent” describes reducing or eliminating the onset of the symptoms or complications of the disease, condition or disorder.

The term “disorder” is used in this application to mean, and is used interchangeably with, the terms disease, condition, or illness, unless otherwise indicated.

As used herein, the term “BTK-mediated” diseases or disorders means any disease or other deleterious condition in which BTK, or a mutant thereof, is known to play a role. Accordingly, another embodiment of the present application relates to treating or lessening the severity of one or more diseases in which BTK, or a mutant thereof, is known to play a role. Specifically, the present application relates to a method of treating or lessening the severity of a disease or condition selected from a proliferative disorder or an autoimmune disorder, wherein said method comprises administering to a patient in need thereof a compound of Formula (I), or pharmaceutically acceptable salts thereof, or a composition according to the present application.

As used herein, the term “cell proliferative disorder” refers to conditions in which unregulated or abnormal growth, or both, of cells can lead to the development of an unwanted condition or disease, which may or may not be cancerous. Exemplary cell proliferative disorders of the application encompass a variety of conditions wherein cell division is deregulated. Exemplary cell proliferative disorder include, but are not limited to, neoplasms, benign tumors, malignant tumors, pre-cancerous conditions, in situ tumors, encapsulated tumors, metastatic tumors, liquid tumors, solid tumors, immunological tumors, hematological tumors, cancers, carcinomas, leukemias, lymphomas, sarcomas, and rapidly dividing cells. The term “rapidly dividing cell” as used herein is defined as any cell that divides at a rate that exceeds or is greater than what is expected or observed among neighboring orjuxtaposed cells within the same tissue.

A cell proliferative disorder includes a precancer or a precancerous condition. A cell proliferative disorder includes cancer. Preferably, the methods provided herein are used to treat or alleviate a symptom of cancer. The term “cancer” includes solid tumors, as well as hematologic tumors and/or malignancies. A “precancer cell” or “precancerous cell” is a cell manifesting a cell proliferative disorder that is a precancer or a precancerous condition. A “cancer cell” or “cancerous cell” is a cell manifesting a cell proliferative disorder that is a cancer. Any reproducible means of measurement may be used to identify cancer cells or precancerous cells. Cancer cells or precancerous cells can be identified by histological typing or grading of a tissue sample (e.g., a biopsy sample). Cancer cells or precancerous cells can be identified through the use of appropriate molecular markers.

Exemplary non-cancerous BTK-mediated conditions or disorders are referenced in U.S. Pat. No. 9,630,968, incorporated herein by reference.

### Method for Preparing the Compound of Formula I

The compound of Formula I may be made by a variety of methods, including standard chemistry. The compound of Formula (I) may be prepared by methods known in the art of organic synthesis as described in U.S. Pat. No. 9,630,968, incorporated herein by reference.

The compounds described herein may be made from commercially available starting materials or synthesized using known organic, inorganic, and/or enzymatic processes.

The compounds of the present application can be prepared in a number of ways well known to those skilled in the art of organic synthesis.

### Methods of Using the Compounds

Aspect of the application relates to a method of treating, preventing, inhibiting, or eliminating a disease or disorder associated with modulation of BTK (e.g., inhibition of BTK). The method comprises administering to a patient in need of a treatment for diseases or disorders associated with modulation of BTK an effective amount the compound of Formula (I), or a pharmaceutically acceptable salt thereof or a pharmaceutical composition of the compound of Formula (I), in combination with a second agent as described herein. In one embodiment, the BTK-mediated disorder is selected from immune disorders, cancer, cardiovascular diseases, viral infections, inflammation, metabolism/endocrine function disorders and neurological disorders.

In embodiments, the BTK is mutant BTK (e.g., BTK C481S mutant).

An aspect of the invention relates to methods that comprises administering at least one second agent selected from an anti-inflammatory agent, an immunomodulatory agent, chemotherapeutic agent, a neurotropic factor, an agent for treating cardiovascular disease, an agent for treating liver disease, an anti-viral agent, an agent for treating blood disorders, an agent for treating diabetes, and an agent for treating immunodeficiency disorders.

Another aspect of the application relates to a method of modulating BTK, the method comprising administering to a patient in need thereof a therapeutically effective amount of the compound of Formula (I), or a pharmaceutically acceptable salt thereof or a pharmaceutical composition of the compound of Formula (I) in combination with a second agent as described herein. In one embodiment, modulating BTK is inhibiting BTK.

Another aspect of the application relates to the compound of Formula (I), or a pharmaceutically acceptable salt thereof, in combination with a second agent as described herein, for use in a method of treating a BTK-mediated disorder. In one embodiment, the disease or disorder is selected from immune disorders, cancer, cardiovascular diseases, viral infections, inflammation, metabolism/endocrine function disorders and neurological disorders.

In embodiments, the methods described herein comprise administering at least one second agent which is selected from an anti-inflammatory agent, an immunomodulatory agent, chemotherapeutic agent, a neurotropic factor, an agent for treating cardiovascular disease, an agent for treating liver disease, an anti-viral agent, an agent for treating blood disorders, an agent for treating diabetes, and an agent for treating immunodeficiency disorders.

In some embodiments, the BTK is wild-type BTK. In other embodiments, the BTK is mutant BTK (e.g., BTK C481S mutant).

In another aspect, the present application relates to a pharmaceutical composition of the compound of Formula (I), or a pharmaceutically acceptable salt thereof in combination with a second agent as described herein, for use in a method of treating a BTK-mediated disorder.

Another aspect of the application relates to the compound of Formula (I), or a pharmaceutically acceptable salt thereof in combination with a second agent as described herein, for use in a method of treating, preventing, inhibiting, or eliminating a cell proliferative disorder.

In one embodiment, the cell proliferative disorder is a cancer.

In another aspect. the present application relates to a pharmaceutical composition of the compound of Formula (I), or a pharmaceutically acceptable salt thereof, in combination with a second agent as described herein, for use in a method of treating, preventing, inhibiting, or eliminating a cell proliferative disorder. In one embodiment, the cell proliferative disorder is a cancer.

In some embodiments of the methods and uses described herein, the cancer is selected from breast, ovary, cervix, prostate, testis, genitourinary tract, esophagus, larynx, glioblastoma, neuroblastoma, stomach, skin, keratoacanthoma, lung, epidermoid carcinoma, large cell carcinoma, non-small cell lung carcinoma (NSCLC), small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, pancreatic, myeloid disorders, lymphoma, hairy cells, buccal cavity, naso-pharyngeal, pharynx, lip, tongue, mouth, small intestine, colon-rectum, large intestine, rectum, brain and central nervous system, Hodgkin's leukemia, bronchus, thyroid, liver and intrahepatic bile duct, hepatocellular, gastric, glioma/glioblastoma, endometrial, melanoma, kidney and renal pelvis, urinary bladder, uterine corpus, uterine cervix, multiple myeloma, acute myelogenous leukemia, chronic myelogenous leukemia, lymphocytic leukemia, chronic lymphocytic leukemia (CLL), small lymphocytic lymphoma (SLL), B-cell non-Hodgkin lymphoma (e.g., diffuse large B-cell lymphoma (DLBCL), follicular lymphoma, Mantle cell lymphoma (MCL), CNS lymphoma (primary and secondary)), marginal zone lymphoma, myeloid leukemia, pediatric sarcoma, pediatric brain tumors, oral cavity and pharynx, non-Hodgkin lymphoma, melanoma, villous colon adenoma, Richter's transformation and waldenstrom's macroglobulinemia. The terms “CLL” and “SLL” refer to the same disease that is located in a different anatomic location. CLL and SLL have the same treatment paradigms and expected clinical outcomes. CLL is sometimes used as an encompassing term for both CLL and SLL.

In any of the embodiments of the application, the cancer can be any cancer in any organ, for example, a cancer is selected from the group consisting of glioma, thyroid carcinoma, breast carcinoma, small-cell lung carcinoma, non-small-cell carcinoma, gastric carcinoma, colon carcinoma, gastrointestinal stromal carcinoma, pancreatic carcinoma, bile duct carcinoma, CNS carcinoma, ovarian carcinoma, endometrial carcinoma, prostate carcinoma, renal carcinoma, anaplastic large-cell lymphoma, leukemia, multiple myeloma, mesothelioma, and melanoma, and combinations thereof.

In any of the embodiments of the invention, the cancer is chronic lymphocytic leukemia (CLL), small lymphocytic lymphoma (SLL) or Mantle cell lymphoma (MCL). In any of the embodiments, the cancer is chronic lymphocytic leukemia (CLL).

In some embodiments of the methods and uses described herein, the disease or disorder is an immune disorder. In one embodiment, the immune disorder is rheumatoid arthritis.

In some embodiments of the methods and uses described herein, the disease or disorder is systemic and local inflammation, arthritis, inflammation related to immune suppression, organ transplant rejection, allergies, ulcerative colitis, Crohn's disease, dermatitis, asthma, systemic lupus erythematosus, Sjogren's Syndrome, multiple sclerosis, scleroderma/systemic sclerosis, idiopathic thrombocytopenic purpura (ITP), anti-neutrophil cytoplasmic antibodies (ANCA) vasculitis, chronic obstructive pulmonary disease (COPD), psoriasis.

In one embodiment, methods of treating a disease or disorder associated with modulation of BTK including, immune disorders, cancer, cardiovascular diseases, viral infections, inflammation, metabolism/endocrine function disorders and neurological disorders, comprise administering to a patient suffering from at least one of said diseases or disorder the compound of Formula (I) in combination with at least one second agent.

The compound of Formula (I) can be administered in therapeutically effective amounts in a combinational therapy with one or more therapeutic agents (pharmaceutical combinations) or modalities, e.g., non-drug therapies. For example, synergistic effects can occur with other anti-proliferative, anti-cancer, immunomodulatory or anti-inflammatory substances. In some embodiments, the compound of Formula (I) is administered in combination with at least one second agent selected from an anti-inflammatory agent, an immunomodulatory agent, chemotherapeutic agent, a neurotropic factor, an agent for treating cardiovascular disease, an agent for treating liver disease, an anti-viral agent, an agent for treating blood disorders, an agent for treating diabetes, and an agent for treating immunodeficiency disorders. Where the compound of Formula (I) is administered in conjunction with other therapies, dosages of the co-administered compounds will of course vary depending on the type of co-drug employed, on the specific drug employed, on the condition being treated and so forth.

Combination therapy includes the administration of the Compound (I) in further combination with at least one second agent (biologically active ingredients such as, but not limited to, an anti-inflammatory agent, an immunomodulatory agent, chemotherapeutic agent, a neurotropic factor, an agent for treating cardiovascular disease, an agent for treating liver disease, an anti-viral agent, an agent for treating blood disorders, an agent for treating diabetes, and an agent for treating immunodeficiency disorders.

In an embodiment, the compound of Formula (I) is used in combination with at least one second agent, preferably compounds that are able to enhance the effect of the compound of Formula (I). The compound of Formula (I) can be administered simultaneously (as a single preparation or separate preparation) or sequentially to the second agent. In general, a combination therapy envisions administration of two or more drugs during a single cycle or course of therapy. In embodiments of the invention, the methods comprise administration of a therapeutically effective amount of the compound of Formula (I), or a pharmaceutically acceptable salt thereof. prior to the administration of a therapeutically effective amount of at least one second agent, or a pharmaceutically acceptable salt thereof. In embodiment of this invention, the co-administration of the compound of Formula (I) and a second agent occurs within 1 hour, 2 hours, 3 hours, 4, hours, 5 hours, 6 hours, 7 hours, 8 hours, 9 hours, 10 hours, 11 hour, 12 hours, 13 hours, 14. hours, 15 hours, 16 hours, 17 hours, 18 hours. 19 hours, 20 hours, 21 hours, 22 hours, 23 hours or 24 hours. In embodiment of this invention, the co-administration of the compound of Formula (I) and a second agent occurs within 1 day, 2 days, 3 days, 4 days, 5 days, 6 days or 7 days. In embodiment of this invention, the co-administration of the compound of Formula (I) and a second agent occurs within 1 week, 2 weeks, 3 weeks or 4 weeks.

A second agent can include, but is not limited to, an alkylating agent, an antibiotic, an antibiotic anti-neoplastic, an anti-metabolite, an anti-metabolite anti-neoplastic, a detoxifying agent, an interferon, a polyclonal or monoclonal antibody, a Bcl-2 inhibitor, a BTK inhibitor, an mTOR kinase inhibitor, an immunomodulatory drug (ImiD), a TOPK inhibitor, a sphingosine-1-phosphate receptor agonist, an anti-T cell immunoglobulin, an anti-IL-2 receptor antibody, an amide, an EGFR inhibitor, a HER2 inhibitor, a histone deacetylase inhibitor, a hormone or hormone analogue, a mitotic inhibitor, a kinase inhibitor, a multi-kinase inhibitor, a serine/threonine kinase inhibitor, a tyrosine kinase inhibitor, a VEGF/VEGFR inhibitor, a taxane or taxane derivative, an aromatase inhibitor, an anthracycline, a microtubule targeting drug, an anti-microtubule agent, an anti-mitotic agent, a mitotic inhibitor, a topoisomerase poison drug, an inhibitor of a molecular target or enzyme (e.g., a kinase inhibitor), a cytidine analogue, a phosphotidyl inositol-3 kinase (PI3K) inhibitor, an antibody antagonist to receptor kinase ligand binding, an anti-angiogenic therapeutic agent, a SH2/SH3 domain blocker, a myo-inositol signaling inhibitor, a Ras oncogene inhibitor, an anti-neoplastic agent, an anti-proliferative agent, an additional therapeutic agent, an anti-viral agent, an antigen or adjuvant, a cytotoxic agent, a retinoid, a chemotherapeutic agent, a cell cycle signaling inhibitor, a therapeutic agent used in proapoptotic regimes, a PARP inhibitor, an agent used in immunotherapeutic regimens, a checkpoint inhibitor, a PD-1 or PD-L1 inhibitor, an anti-PD-L1 antibody, a PD-1 antagonist, a PD-1 inhibitor, an antibody to ICOS, an antibody to OX040, a TLR agonist, a TLR4 agonist, a therapeutic agent useful in the prevention or treatment of bacterial and viral infections, a therapeutic agent useful in the treatment of TB infection (Mycobacterium tuberculosis) and Tularemia (Franciseiia tularensis), a therapeutic agent useful in the treatment of chlamydia, a therapeutic agent useful in the treatment of plasmodium infection, a vaccine, an immugenic antigen, an anti-inflammatory agent, or any chemotherapeutic, anti-neoplastic or anti-proliferative agent listed in www.cancer.org/docroot/cdg/cdg_0.asp.

B-cell lymphoma 2 protein (Bcl-2) is an important regulator of programmed cell death (apoptosis). Bcl-2 inhibitors can include, but are not limited to: Venetoclax, BI-97C1, sabutoclax, navitoclax, obatoclax, 4-[4-[[2-(4-Chlorophenyl)phenyl]methyl]piperazin-1-yl]-N-[4-[[(2R)-4-(dimethylamino)-1-phenylsulfanylbutan-2-yl]amino]-3-nitrophenyl]sulfonylbenzamide (ABT-737), N-[4-(2-tert-butylphenyl)sulfonylphenyl]-2,3,4-trihydroxy-5-[(2-propan-2-ylphenyl)methyl]benzamide (TW-37), APG-1252 (Ascentage Pharma), APG-2575 (Ascentage Pharma) and S 55746 (Servier). Bcl-2 inhibitors can also include, but are not limited to other chemical entities, such as antisense RNAs, siRNAs, peptidyl inhibitors and antibody inhibitors of Bcl-2. Bcl-2 inhibitors can also include, but are not limited to inhibitors of other members of the Bcl-2-mediated signaling pathway may be used in lieu of or in addition to Bcl-2 inhibitors. In an embodiment of the instant invention, the second agent is a Bcl-2 inhibitor. In an embodiment of the instant invention, the second agent is venetoclax.

Exemplary BTK inhibitors can include, but are not limited to, ibrutinib, ACP-196 (acalabrutinib), BGB-3111 (zanubrutinib), spebrutinib, ONO-4059, HM71224, RN486, 4-(4-((4-((3-acrylamidophenyl)amino)-5-fluoropyrimidin-2-yl)amino)phenoxy)-N-methylpicolinamide (CNX-774), N-[3-[4,5-dihydro-4-methyl-6-[[4-(4-morpholinylcarbonyl)phenyl]amino]-5-oxopyrazinyl]-2-methylphenyl]-4-(1,1-dimethylethyl)-benzamide (CGI-1746), CGI-560, AVL-292 (CC-292), PRN1008, M7583, M2951, BIIB068, CT-1530, ACO0058TA, GS-4059, REDX08608, RXC005, BMS-986142, TP-0158, SNS-062, BI-BTK-1, GDC-0834, GDC-0853, 30 LFM-A13, LOU684, PRN1008, acalabrutinib, zanubrutinib and PRN2246. BTK inhibitors can include, but are not limited to polyfluorinated compounds described in PCT Publication WO 2015/165279 and U.S. application Ser. No. 15/075,033 the disclosure of which are incorporated by reference herein in their entireties. BTK inhibitors can include, but are not limited to other chemical entities, such as antisense RNAs, siRNAs, peptidyl inhibitors and antibody inhibitors of BTK. In some embodiments, a BTK inhibitor can bind irreversibly to BTK.

In some embodiments, a BTK inhibitor can bind to mutated BTK, such as BTK with a mutation at C481, e.g., a C481S mutation. In some embodiments, BTK inhibitors can include, but are not limited to, inhibitors of other members of the BTK-mediated signaling pathway. Inhibitors of other members of the BTK-mediated signaling pathway can include, but are not limited to, inhibitors of Protein Kinase C (PKC) R such as enzastaurin and sotrastaurin. In some embodiments, the BTK inhibitor is ibrutinib, acalabrutinib, or zanubrutinib.

Immunomodulatory drugs (IMiDs) are useful in treating a variety of inflammatory and autoimmune diseases. IMiDs also are useful in treating neoplastic diseases such as hematologic neoplasms, e.g., multiple myeloma and myelodysplastic syndromes, as well as certain solid tumors. Exemplary immunomodulatory (IMiDs) drugs can include, but are not limited to lenalidomide, pomalidomide, thalidomide, revlimid, CC-112 and CC-220.

The mammalian target of rapamycin (mTOR) is a protein kinase that serves as a key regulator of cell growth, proliferation, metabolism and apoptosis. Exemplary mTOR Kinase inhibitors can include, but are not limited to, everolimus, rapamycin, [7-(6-Amino-3-pyridinyl)-2,3-dihydro-1,4-benzoxazepin-4(5H)-yl][3-fluoro-2-methyl-4-(methylsulfonyl)phenyl]-methanone (XL388), N-ethyl-N′-[4-[5,6,7,8-tetrahydro-4-[(3S)-3-methyl-4-morpholinyl]-7-(3-oxetanyl)pyrido[3,4-d]pyrimidin-2-yl]phenyl]-Urea (GDC-0349 (Genentech)), 3-(2,4-bis((S)-3-methylmorpholino)pyrido[2,3-d]pyrimidin-7-yl)-N-methylbenzamide (AZD2014 (AstraZeneca)), (5-(2,4-bis((S)-3-methylmorpholino)pyrido[2,3-d]pyrimidin-7-yl)-2-methoxyphenyl)methanol (AZD8055), GSK105965, 3-(2-aminobenzo[d]oxazol-5-yl)-1-isopropyl-1H-pyrazolo[3,4-d]pyrimidin-4-amine (TAK-228 or MLN0128 (Takeda)), temsirolimus. ridaforolimus, P1-103, NVP-BEZ235, WJD008, XL765, SF-1126, Torin1, PP242, PP30, Ku-0063794, WYE-354, WYE-687, WAY-600, INK128, OSI-027, gedatolisib or PF-05212384 (Pfizer), CC-223 (Celgene), LY3023414 (Lilly), PQR309 (PIQUR Therapeutics), LXI-15029 (Luoxin Pharma) and SAR245409 (Sanofi). Exemplary mTOR kinase inhibitors can also include, but are not limited to other chemical entities, such as antisense RNAs, siRNAs, peptidyl inhibitors and antibody inhibitors of mTOR. Exemplary mTOR kinase inhibitors can also include, but are not limited to, inhibitors of other members of the mTOR-mediated signaling pathway. Inhibitors of other members of the mTOR-mediate signaling pathway can include, but are not limited to, inhibitors of phosphoinositide 3-kinase (P13K) such as BTG226, gedatolisib, apitolisib, omipalisib, dactolisib, duvelisib (COPIKTRA), idelalisib (Zydelig), temsirolimus (Torisel), rapamune, ridaforolimus, and AP23573, as well as inhibitors of Akt (Protein Kinase B) such as 8-[4-(1-aminocyclobutyl)phenyl]-9-phenyl-2H-[1,2,4]triazolo[3,4-f][1,6]naphthyridin-3-one;dihydrochloride (MK-2206).

Exemplary TOPK inhibitors include, but are not limited to, OTS964 ((R)-9-(4-(1-(dimethylamino)propan-2-yl)phenyl)-8-hydroxy-6-methylthieno[2,3-c]quinolin-4(5H)-one).

Exemplary sphingosine-1-phosphate receptor agonists can include, but are not limited to, fingolimod and KRP-203.

Exemplary anti-T cell immunoglobulins can include, but are not limited to, anti-thymocyte globulin (Atgam®)

Exemplary anti-IL-2 receptor antibodies can include, but are not limited to daclizumab.

Exemplary amides can include, but are not limited to, CTX.

Exemplary second agents can also include, but are not limited to, ifosfamide (IFO), adriamycin (ADM), daunorubicin (DNR), vincristine (VCR), vinblastine (VBL), etoposide (VP16), vermeer (Vumon), carboplatin (CBP) and methotrexate (MTX) cyclosporin A, tacrolimus, sirolimus, everolimus, azathioprine, brequinar, leflunomide, LEA-29Y, anti-CD3 antibody (e.g. OKT3), aspirin, B7-CD28 blocking molecules (e.g. belatacept, abatacept), CD40-CD154 blocking molecules (anti-CD40 antibodies), acetaminophen, ibuprofen, naproxen, piroxicam, and anti-inflammatory steroids (e.g. prednisolone or dexamethasone).

Alkylating agents are non-phase specific agents and strong electrophiles. Typically, alkylating agents form covalent linkages, by alkylation, to DNA through nucleophilic moieties of the DNA molecule such as phosphate, amino, sulfhydryl, hydroxy, carboxyl, and imidazole groups. Such alkylation disrupts nucleic acid function leading to cell death. Examples of alkylating agents include, but are not limited to, nitrogen mustards such as cyclophosphamide (e.g., CYTOXAN®), melphalan (e.g., ALKERAN®), and chlorambucil (e.g., LEUKERAN®); alkyl sulfonates such as busulfan (e.g., MYLERAN®); nitrosoureas such as carmustine (e.g., BiCNU®); and triazenes such as dacarbazine (e.g., DTIC-Dome®).

Exemplary alkylating agents also include, but are not limited to, busulfan (Busulfex), lomustine (CeeNU), oxaliplatin (Eloxatin), carmustine (Gliadel), ifosfamide (Ifex), mechlorethamine (Mustargen), busulfan (Myleran), carboplatin (PARAPLATIN®), cisplatin (CDDP, PLATINOLR), temozolomide (Temodar), thiotepa (Thioplex), bendamustine (Treanda), streptozocin (Zanosar), 5-azacytidine (e.g., VIDAZA), decitabine (e.g., DECOGEN), temozolomide (e.g., TEMODAR and TEMODAL), dactinomycin (also known as actinomycin-D and sold under the tradename COSMEGEN), melphalan (also known as L-PAM, L-sarcolysin, and phenylalanine mustard, sold under the tradename ALKERAN), altretamine (also known as hexamethylmelamine (HMM), sold under the tradename HEXALEN), carmustine (e.g., BCNU, BiCNU), bendamustine (e.g., TREANDA), carboplatin (e.g., PARAPLATIN®), lomustine (also known as CCNU, sold under the tradename CeeNUk), cisplatin (also known as CDDP, sold under the tradenames PLATINOL® and PLATINOL®-AQ), cyclophosphamide (sold under the tradenames CYTOXAN® and NEOSAR®), dacarbazine (also known as DTIC, DIC and imidazole carboxamide, sold under the tradename DTIC-DOME®), altretamine (also known as hexamethylmelamine (HMM) sold under the tradename HEXALEN®), ifosfamide (e.g., IFEXR), procarbazine (e.g., MATULANE®), mechlorethamine (also known as nitrogen mustard, mustine and mechloroethamine hydrochloride, sold under the tradename MUSTARGEN®), streptozocin (e.g., ZANOSAR®), and thiotepa (also known as thiophosphoamide, TESPA and TSPA, and sold under the tradename THIOPLEX®).

Antibiotic anti-neoplastics are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin (e.g., COSMEGEN®), anthracyclines such as daunorubicin (e.g., as a liposomal injectable form as DAUNOXOME® or as an injectable as CERUBIDINER) and doxorubicin (e.g., RUBEX® or ADRIAMYCIN RDF®), and bleomycins (e.g., BLENOXANE®). Exemplary anthracyclines include, but are not limited to, daunorubicin (Daunomycin), doxorubicin (Adriamycin), epirubicin, idarubicin, and valrubicin.

Exemplary antibiotics also include, but are not limited to, doxorubicin (Adriamycin), doxorubicin liposomal (Doxil), mitoxantrone (Novantrone), bleomycin (Blenoxane), daunorubicin (Cerubidine), daunorubicin liposomal (DaunoXome), dactinomycin (Cosmegen), epirubicin (Ellence), idarubicin (Idamycin), plicamy cin (Mithracin), mitomycin (Mutamycin), pentostatin (Nipent), valrubicin (Valstar), doxorubicin (e.g., ADRIAMYCIN® and RUBEX®), bleomycin (e.g., LENOXANE®), daunorubicin (also known as dauorubicin hydrochloride, daunomycin, and rubidomycin hydrochloride, sold under the tradename CERUBIDINE®), daunorubicin liposomal (daunorubicin citrate liposome, sold under the tradename DAUNOXOME), mitoxantrone (also known as DHAD, sold under the tradename NOVANTRONE®), epirubicin (e.g., ELLENCE™), idarubicin (e.g., IDAMYCIN®, IDAMYCIN PFS®), and mitomycin C (e.g., MUTAMYCIN®).

Anti-metabolite anti-neoplastic agents are phase specific agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil and analogs thereof (e.g., 5-fluoro deoxyuridine (floxuridine), 5-fluorodeoxyuridine monophosphate methotrexate), cytarabine (commonly known as Ara-C, available as CYTOSAR-U®) and analogs thereof (e.g., azacytidine, 2′,2′-difluorodeoxycytidine (gemcitabine)), mercaptopurine (e.g., PURINETHOL®) and analogs thereof (e.g., azathioprine), thioguanine (e.g., TABLOID®) and analogs thereof (e.g., pentostatin, erythrohydroxy-nonyladenine (EHNA), fludarabine phosphate, and cladribine), gemcitabine (e.g., GEMZARR), and methotrexate.

Exemplary anti-metabolites also include, but are not limited to, fluorouracil (Adrucil), capecitabine (Xeloda), hydroxyurea (Hydrea), mercaptopurine (Purinethol), pemetrexed (Alimta), fludarabine (Fludara), nelarabine (Arranon), cladribine (Cladribine Novaplus), clofarabine (Clolar), cytarabine (Cytosar-U), decitabine (Dacogen), cytarabine liposomal (DepoCyt), hydroxyurea (Droxia), pralatrexate (Folotyn), floxuridine (FUDR), gemcitabine (Genzar), cladribine (Leustatin), fludarabine (Oforta), methotrexate (MTX, Rheumatrex), methotrexate (Trexall), thioguanine (Tabloid), TS-1 or cytarabine (Tarabine PFS), claribine (2-chlorodeoxyadenosine, sold under the tradename LEUSTATIN®), 5-fluorouracil (sold under the tradename ADRUCIL®), 6-thioguanine (sold under the tradename PURINETHOL®), pemetrexed (sold under the tradename ALIMTA®), cytarabine (also known as arabinosylcytosine (Ara-C), sold under the tradename CYTOSAR-UR), cytarabine liposomal (also known as Liposomal Ara-C, sold under the tradename DEPOCYT^{IM}), decitabine (sold under the tradename DACOGENR), hydroxyurea and (sold under the tradenames HYDREAR, DROXIA™ and MYLOCEL™), fludarabine (sold under the tradename FLUDARA®), floxuridine (sold under the tradename FUDR®), cladribine (also known as 2-chlorodeoxyadenosine (2-CdA) sold under the tradename LEUSTATIN™), methotrexate (also known as amethopterin, methotrexate sodium (MTX), sold under the tradenames RHEUMATREX® and TREXALL™), and pentostatin (sold under the tradename NIPENT®).

Exemplary detoxifying agents include, but are not limited to, amifostine (Ethyol), and mesna (Mesnex).

Exemplary interferons include, but are not limited to, interferon alfa-2b (Intron A), and interferon alfa-2a (Roferon-A).

Exemplary polyclonal or monoclonal antibodies include, but are not limited to, trastuzumab (Herceptin), ofatumumab (Arzerra), bevacizumab (Avastin), rituximab (Rituxan), cetuximab (Erbitux), panitumumab (Vectibix), tositumomab/iodine¹³¹ tositumomab (Bexxar), alemtuzumab (Campath), ibritumomab (Zevalin, In-111, Y-90 Zevalin), gemtuzumab (Mylotarg), eculizumab (Soliris), ordenosumab, denosumab, and anti-CD20 antibodies (e.g., rituximab (Rituxan), obinutuzumab (Gazyva), ofatumumab (Arzerra), and ublituximab).

Exemplary EGFR inhibitors include, but are not limited to, gefitinib (Iressa), lapatinib (Tykerb), cetuximab (Erbitux), erlotinib (Tarceva), panitumumab (Vectibix), PKI-166, canertinib (CI-1033), matuzumab (Emd7200), and EKB-569.

Exemplary HER2 inhibitors include, but are not limited to, trastuzumab (Herceptin); lapatinib (Tykerb), and AC-480.

Exemplary histone deacetylase Inhibitors include, but are not limited to, vorinostat (Zolinza).

Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, adrenocorticosteroids such as prednisone and prednisolone; aminoglutethimide and other aromatase inhibitors such as, but not limited to, anastrozole, letrozole, vorozole, and exemestane; progestins such as megestrol acetate; estrogens, and anti-estrogens such as fulvestrant, flutamide, nilutamide, bicalutamide, cyproterone acetate and 5-reductases such as finasteride and dutasteride; anti-estrogens such as tamoxifen, toremifene, raloxifene, droloxifene, iodoxyfene, as well as selective estrogen receptor modulators (SERMS) such those described in U.S. Pat. Nos. 5,681,835, 5,877,219, and 6,207,716; and gonadotropin-releasing hormone (GnRH) and analogues thereof; and LHRH agonists and antagonists such as goserelin acetate and luprolide.

Exemplary aromatase inhibitors include, but are not limited to, aminoglutethimide, testolactone (Teslac), anastrozole (Arimidex), Letrozole (Femara), exemestane (Aromasin), Vorozole (Rivizor), Formestane (Lentaron), Fadrozole (Afema), 4-androstene-3,6,17-trione (6-OXO), 1,4,6-androstatrien-3,17-dione (ATD), and 4-hydroxyandrostenedione.

Exemplary hormones also include, but are not limited to, tamoxifen (Soltamox, Nolvadex), raloxifene (Evista), megestrol (Megace), leuprolide (Lupron, Lupron Depot, Eligard, Viadur), fulvestrant (Faslodex), letrozole (Femara), triptorelin (Trelstar L A, Trelstar Depot), exemestane (Aromasin), goserelin (Zoladex), bicalutamide (Casodex), anastrozole (Arimidex), fluoxymesterone (Androxy, Halotestin), medroxyprogesterone (Provera, Depo-Provera), estramustine (Emcyt), flutamide (Eulexin), toremifene (Fareston), degarelix (Firmagon), nilutamide (Nilandron), abarelix (Plenaxis), or testolactone (Teslac).

Anti-microtubule or anti-mitotic agents or mitotic inhibitors are phase specific agents active against the microtubules of tumor cells during M or the mitosis phase of the cell cycle. Examples of anti-microtubule agents include, but are not limited to, diterpenoids and vinca alkaloids. Examples of diterpenoids include, but are not limited to, paclitaxel (e.g., TAXOL®) and its analog docetaxel (e.g., TAXOTERE®). Examples of vinca alkaloids include, but are not limited to, vinblastine (e.g., VELBAN®), vincristine (e.g., ONCOVIN®), and vinorelbine (e.g., NAVELBINE®).

Exemplary mitotic inhibitors also include, but are not limited to, paclitaxel (Taxol, Onxol, Abraxane), docetaxel (Taxotere), vincristine (Oncovin, Vincasar PFS), vinblastine (Velban), etoposide (Toposar, Etopophos, VePesid), teniposide (Vumon), ixabepilone (Ixempra), nocodazole, epothilone, vinorelbine (Navelbine), camptothecin (CPT), irinotecan (Camptosar), topotecan (Hycamtin), amsacrine, and lamellarin D (LAM-D).

Exemplary multi-kinase inhibitors include, but are not limited to, sorafenib (Nexavar), sunitinib (Sutent), BIBW 2992, E7080, Zd6474, PKC-412, motesanib, and AP24534.

Exemplary serine/threonine kinase inhibitors include, but are not limited to, ruboxistaurin, eril/easudil hydrochloride, flavopiridol, seliciclib (CYC202, Roscovitrine), SNS-032 (BMS-387032), Pkc412, bryostatin, KAI-9803, SF1126, VX-680, Azd1152, Arry-142886 (AZD-6244), SCIO-469, GW681323, CC-401, CEP-1347, and PD 332991.

Exemplary serine/threonine kinase inhibitors include, but are not limited to, Rapamune (targets mTOR/FRAP1); Deforolimus (targets mTOR); Certican/Everolimus (targets mTOR/FRAP1); AP23573 (targets mTOR/FRAP1); Eril/Fasudil hydrochloride (targets RHO); Flavopiridol (targets CDK); Seliciclib/CYC202/Roscovitrine (targets CDKs); SNS-032/BMS-387032 (targets CDKs); Ruboxistaurin (targets PKC); Pkc412 (targets PKC); Bryostatin (targets PKC); KAI-9803 (targets PKC); SF1126 (targets PI3K); VX-680 (targets Aurora kinase); Azd1152 (targets Aurora kinase); Arry-142886/AZD-6244 (targets MAP/MEK); SCIO-469 (targets MAP/MEK); GW681323 (targets MAP/MEK); CC-401 (targets JNK); CEP-1347 (targets JNK); and PD 332991 (targets CDKs).

Inhibitors of serine/threonine kinases also include MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta), IkB kinase family (IKKa, IKKb), PKB family kinases, AKT kinase family members, and TGF beta receptor kinases. Inhibitors of serine/threonine kinases are also described in J. Biochem. 126, 799 (1999), Biochem. Pharmacol. 60, 1101 (2000); Cancer Surveys 27, 41 (1996); Cancer Treatment Res. 78, 3 (1995); Bioorg. Med. Chem. Letters 10, 223 (2000); U.S. Pat. No. 6,268,391; and Int. J. Cancer 88, 44 (2000).

Tyrosine kinases (also nonreceptor tyrosine kinases) include, but are not limited to, cSrc, Lek, Fyn, Yes, Jak, cAbl, FAK (Focal adhesion kinase), Brutons tyrosine kinase, and Bcr-Abl. Inhibitors of non-receptor tyrosine kinase are described in J Hematotherapy Stem Cell Res. 8, 465 (1999) and Annual Rev. Immunol. 15, 371 (1997).

Exemplary tyrosine kinase inhibitors also include, but are not limited to, erlotinib (Tarceva), gefitinib (Iressa), imatinib (Gleevec), sorafenib (Nexavar), sunitinib (Sutent), trastuzumab (Herceptin), bevacizumab (Avastin), rituximab (Rituxan), lapatinib (Tykerb), cetuximab (Erbitux), panitumumab (Vectibix), everolimus (Afinitor), alemtuzumab (Campath), gemtuzumab (Mylotarg), temsirolimus (Torisel), pazopanib (Votrient), dasatinib (Sprycel), nilotinib (Tasigna), vatalanib (Ptk787, ZK222584), icotinib, CEP-701, SU5614, MLN518, XL999, VX-322, Azd0530, BMS-354825, SKI-606 CP-690, AG-490, WHI-P154, WHI-P131, AC-220, and AMG888.

Exemplary VEGF/VEGFR inhibitors include, but are not limited to, bevacizumab (Avastin), sorafenib (Nexavar), sunitinib (Sutent), ranibizumab, pegaptanib, and vandetinib, axitinib, brivanib alaninate ((S)-((R)-1-(4-(4-Fluoro-2-methyl-1H-indol-5-yloxy)-5-methylpyrrolo[2, 1-f][1,2,4]triazin-6-yloxy)propan-2-yl)2-aminopropanoate, also known as BMS-582664), motesanib (TST-(2,3-dihydro-3,3-dimethyl-1H-indol-6-yl)-2-[(4-pyridinylmethyl)amino]-3-pyridinecarboxamide), and pasireotide (also known as SO 230).

Inhibitors of phosphotidyl inositol-3 kinase family members including blockers of Pekinese, ATM, DNA-PK, and Ku may also be combined with the compounds of the present application. Such inhibitors are discussed in Curr. Opinion Immunol. 8, 412 (1996); Oncogene 17, 3301 (1998); Int. J. Biochem. Cell Biol. 29, 935 (1997); and Cancer Res. 60, 1541 (2000).

Exemplary kinase inhibitors also include, but are not limited to, Bevacizumab (targets VEGF), BIBW 2992 (targets EGFR and Erb2), Cetuximab/Erbitux (targets Erb1), Imatinib/Gleevic (targets Bcr-Abl, PDGFRs and c-Kit), Trastuzumab (targets Erb2), Gefitinib/Iressa (targets EGFR), Ranibizumab (targets VEGF), Pegaptanib (targets VEGF), Erlotinib/Tarceva (targets Erbl). Nilotinib (targets Bcr-Abl), Lapatinib (targets Erbl and Erb2/Her2), GW-572016/lapatinib ditosylate (targets HER2/Erb2), Panitumumab/Vectibix (targets EGFR), Vandetinib (targets RET/VEGFR), E7080 (multiple targets including RET and VEGFR), Herceptin (targets HER2/Erb2), PKI-166 (targets EGFR), Canertinib/CI-1033 (targets EGFR), Sunitinib/SU-11464/Sutent (targets EGFR and FLT3), Matuzumab/Emd7200 (targets EGFR), EKB-569 (targets EGFR), Zd6474 (targets EGFR and VEGFR), PKC-412 (targets VEGR and FLT3), Vatalanib/Ptk787/ZK222584 (targets VEGR), CEP-701 (targets FLT3), SU5614 (targets FLT3), MLN518 (targets FLT3), XL999 (targets FLT3), VX-322 (targets FLT3), Azd0530 (targets SRC), BMS-354825 (targets SRC), SKI-606 (targets SRC), CP-690 (targets JAK), AG-490 (targets JAK), WHI-P154 (targets JAK), WHI-P131 (targets JAK), sorafenib/Nexavar (targets RAF kinase, VEGFR-1, VEGFR-2, VEGFR-3, PDGFR-B, KIT, FLT-3, and RET), Dasatinib/Sprycel (BCR/ABL and Src), AC-220 (targets Flt3), AC-480 (targets all HER proteins, “panHER”), Motesanib diphosphate (targets VEGF1-3, PDGFR, and c-kit), Denosumab (targets RANKL, inhibits SRC), AMG888 (targets HER3), and AP24534 (multiple targets including Flt3).

Exemplary microtubule targeting drugs include, but are not limited to, paclitaxel, docetaxel, vincristin, vinblastin, nocodazole, epothilones and navelbine.

Exemplary topoisomerase poison drugs include, but are not limited to, teniposide, etoposide, adriamycin, camptothecin, daunorubicin, dactinomycin, mitoxantrone, amsacrine, epirubicin, and idarubicin.

Additional topoisomerase poison drugs include topoisomerase II inhibitors, such as epipodophyllotoxins. Examples of epipodophyllotoxins include, but are not limited to, etoposide (VP-16, VePESID®)and teniposide (VM-26, VUMON®).

Exemplary taxanes or taxane derivatives include, but are not limited to, paclitaxel and docetaxol.

Antibody antagonists to receptor kinase ligand binding may also serve as inhibitors. Examples include Imclone C225 EGFR specific antibody (see Cancer Treat. Rev. 26, 269 (2000)), Herceptin® erbB2 antibody (see Breast Cancer Res. 2, 176 (2000)), and 2CB VEGFR2 specific antibody (see Cancer Res. 60, 5117 (2000)).

Anti-angiogenic therapeutic agents including non-receptor MEK angiogenesis inhibitors may also be combined with the compounds of the present application, for example, a compound of Formula (I). Anti-angiogenic agents such as those which inhibit the effects of vascular endothelial growth factor (for example, bevacizumab [Avastin™]) and compounds that work by other mechanisms (for example, linomide, inhibitors of integrin av33 function, endostatin and angiostatin).

SH2/SH3 domain blockers can disrupt SH2 or SH3 domain binding in a variety of enzymes or adaptor proteins including, P13-K p85 subunit, Src family kinases, adaptor molecules (She, Crk, Nek, Grb2), and Ras-GAP. SH2/SH3 domains blockers as anticancer drugs are discussed in J. Pharm. Toxicol. Methods 34, 125 (1995).

Myo-inositol signaling inhibitors can include, but are not limited to, phospholipase C blockers and Myoinositol analogues. Such signal inhibitors are described in New Molecular Targets for Cancer Chemotherapy ed., Paul Workman and David Kerr, CRC press 1994, London.

Inhibitors of Ras oncogene can include, but are not limited to, inhibitors of farnesyltransferase, geranyl-geranyl transferase, and CAAX proteases, as well as anti-sense oligonucleotides, ribozymes and immunotherapy. Such inhibitors are discussed in J. Biomed Sci. 7, 292 (2000); Curr. Opin. Lipidology 9, 99 (1998); and BioChim. Biophys. Acta, 1423, 19 (1989).

Additional exemplary general chemotherapeutic, anti-neoplastic, or anti-proliferative agents also include, but are not limited to, altretamine (Hexalen), isotretinoin (Accutane, Amnesteem, Claravis, Sotret), tretinoin (Vesanoid), azacitidine (Vidaza), bortezomib (Velcade), Selinexor, asparaginase (Elspar), levamisole (Ergamisol), mitotane (Lysodren), procarbazine (Matulane), pegaspargase (Oncaspar), denileukin diftitox (Ontak), porfimer (Photofrin), aldesleukin (Proleukin), lenalidomide (Revlimid), bexarotene (Targretin), thalidomide (Thalomid), temsirolimus (Torisel), arsenic trioxide (Trisenox), verteporfin (Visudyne), mimosine (Leucenol), (IM tegafur -0.4 M 5-chloro-2,4-dihydroxypyrimidine -1 M potassium oxonate) and statins (e.g., lovastatin, atorvastatin, cerivastatin, fluvastatin, mevastatin, pitavastatin, pravastatin, rosuvastatin, and simvastatin).

Anti-viral agents can include, but are not limited to, hepatitis B virus (HBV) inhibitors, hepatitis C virus (HCV) protease inhibitors, HCV polymerase inhibitors, HCV NS4A inhibitors, HCV NS5 A inhibitors, HCV NS5b inhibitors, and human immunodeficiency virus (HIV) inhibitors.

Antigens or adjuvants can include, but not limited to, B7 costimulatory molecule, interleukin-2, interferon-y, GM-CSF, CTLA-4 antagonists, OX-40/OX-40 ligand, CD40/CD40 ligand, sargramostim, levamisol, vaccinia virus, Bacille Calmette-Guerin (BCG), liposomes, alum, Freund's complete or incomplete adjuvant, detoxified endotoxins, mineral oils, surface active substances such as lipolecithin, pluronic polyols, polyanions, peptides, and oil or hydrocarbon emulsions. In one embodiment, adjuvants, such as aluminum hydroxide or aluminum phosphate, can be added to increase the ability of the vaccine to trigger, enhance, or prolong an immune response. In one embodiment, additional materials, such as cytokines, chemokines, and bacterial nucleic acid sequences, like CpG, a toll-like receptor (TLR) 9 agonist as well as additional agonists for TLR 2, TLR 4, TLR 5, TLR 7, TLR 8, TLR9, including lipoprotein, LPS, monophosphoryllipid A, lipoteichoic acid, imiquimod, resiquimod, and in addition retinoic acid-inducible gene I (RIG-I) agonists such as poly I.C, can also be used.

Cytotoxic agents can include, but are not limited to, arsenic trioxide (TRISENOX®), asparaginase (also known as L-asparaginase, and Erwinia L-asparaginase, sold under the tradenames ELSPAR® and KIDROLASE®).

Retinoids can include, but are not limited to, alitretinoin (sold under the tradename PANRETIN®), tretinoin (all-trans retinoic acid, also known as ATRA, sold under the tradename VESANOID®), Isotretinoin (13-c/s-retinoic acid, sold under the tradenames ACCUTANE®, AMNESTEEM®, CLARAVIS®, CLARUS®, DECUTAN®, ISOTANE®, IZOTECH®, ORATANE®, ISOTRET®, and SOTRET®), and bexarotene (sold under the tradename TARGRETIN®).

Chemotherapeutic agents can include, but are not limited to abiraterone acetate, altretamine, anhydrovinblastine, auristatin, bexarotene, bicalutamide, BMS 184476, 2,3,4,5,6-pentafluoro-N-(3-fluoro-4-methoxyphenyl) benzene sulfonamide, bleomycin, N,N-dimethyl-L-valyl-L-valyl-N-methyl-L-valyl-L-prolyl-1-Lproline-t-butylamide, cachectin, cemadotin, chlorambucil, cyclophosphamide, 3′,4′-didehydro-4′deoxy-8′-norvin-caleukoblastine, docetaxol, doxetaxel, cyclophosphamide, carboplatin, carmustine, cisplatin, cryptophycin, cyclophosphamide, cytarabine, dacarbazine (DTIC, DITC-Dome), dactinomycin, daunorubicin, decitabine dolastatin, doxorubicin (adriamycin), etoposide, 5-fluorouracil, finasteride, flutamide, hydroxyurea and hydroxyurea andtaxanes, ifosfamide, liarozole, lonidamine, lomustine (CCNU), MDV3100, mechlorethamine (nitrogen mustard), melphalan, mivobulin isethionate, rhizoxin, sertenef, streptozocin, mitomycin, methotrexate, taxanes, nilutamide, nivolumab, onapristone, paclitaxel, pembrolizumab, prednimustine, procarbazine, RPR109881, stramustine phosphate, tamoxifen, tasonermin, taxol, tretinoin, vinblastine, vincristine, vindesine sulfate, vinflunine, CHOP (i.e., a combination of Cyclophosphamide, Doxorubicin, Vincristine, and Prednisone), EPOH (i.e., a combination of etoposide, prednisolone, oncovin, and hydroxydaunorubicin), EPOCH (i.e., a combination of etoposide, prednisolone, oncovin, cyclophosphamide, and hydroxydaunorubicin). and R-EPOCH (i.e., a combination of rituximab, etoposide, prednisolone, oncovin, cyclophosphamide, and hydroxydaunorubicin) Exemplary cytidine analogs include, but are not limited to, gemcitabine, azacytidine (e.g., 5-azacytidine), and cytosine arabinoside (cytarabin, araC, Cytosar).

Additional therapeutic agents can include, but are not limited to, inhibitors of cell cycle signaling, proapoptotic agents, PARP inhibitors, checkpoint therapeutics, and immune modulators.

Cell cycle signaling inhibitors inhibit molecules involved in the control of the cell cycle. A family of protein kinases called cyclin dependent kinases (CDKs) and their interaction with a family of proteins termed cyclins controls progression through the eukaryotic cell cycle. Several inhibitors of cell cycle signaling are under development. For instance, examples of cyclin dependent kinases, including CDK2, CDK4, and CDK6 and inhibitors for the same are described in Exp. Opin. Ther. Patents 10, 215 (2000).

Therapeutic agents used in proapoptotic regimens (e.g., bcl-2 antisense oligonucleotides) may also be used in combination of the compounds of the present application, for example, a compound of Formula (I).

As used herein, PARP inhibitors refer to a group of pharmacological inhibitors of the enzyme poly ADP ribose polymerase (PARP). Exemplary PARP inhibitors include, but are not limited to, Olaparib (AZD-2281, Lynparza® by Astra Zeneca), Rucaparib (PF-01367338, Rubraca® by Clovis Oncology), Niraparib (MK-4827, Zejula® by Tesaro), Talazoparib (BMN-673), Veliparib (ABT-888), Olaparib (AZD-2281), Olaparib TOPARP-A, Rucaparib (PF-01367338, AG014699), CEP 9722, E7016 (developed by Eisai), BGB-2901, Iniparib (BSI 201), 3-aminobezamide and pamiparib.

Agents used in immunotherapeutic regimens may also be useful in combination with the compounds of the present application, for example, a compound of Formula (I). Immunotherapy approaches, including ex-vivo and in-vivo approaches to increase the immunogenicity of patient tumor cells, such as transfection with cytokines such as interleukin 2, interleukin 4 or granulocyte-macrophage colony stimulating factor, approaches to decrease T-cell energy, approaches using transfected immune cells such as cytokine-transfected dendritic cells, approaches using cytokine transfected tumor cell lines, and approaches using anti-idiotypic antibodies.

As used herein, checkpoint inhibitor therapy refers to a form of cancer treatment immunotherapy that targets immune checkpoints, key regulators of the immune system that stimulate or inhibit its actions, which may allow tumors to protect themselves from attacks by the immune system. Checkpoint therapy can block inhibitory checkpoints, restoring immune system function.

As used herein, “immune-modulators” or “immunomodulators” refer to any substance including monoclonal antibodies that affect the immune system. Immune-modulators and immunomodulators can include, but are not limited to check point inhibitors. Immuno-modulators can be used as anti-neoplastic agents for the treatment of cancer. For example, immune-modulators include, but are not limited to, anti-CTLA-4 antibodies such as ipilimumab (YERVOY) and anti-PD-1 antibodies (Opdivo/nivolumab and Keytruda/pembrolizumab). Other immuno-modulators include, but are not limited to, ICOS antibodies, OX-40 antibodies, PD-L1 antibodies, LAG3 antibodies, TIM-3 antibodies, 41BB antibodies, and GITR antibodies. CLTA-4 and PD-1 pathways are important negative regulators of immune response. Activated T-cells up-regulate CTLA-4, which binds on antigen-presenting cells and inhibits T-cell stimulation, IL-2 gene expression, and T-cell proliferation. PD-1 binds to active T-cells and suppresses T-cell activation. PD-1 antagonists have demonstrated anti-tumor effects. CTLA-4 and PD-1 pathway antagonists that may be used in combination with the compounds of the present application include ipilimumab, tremelimumab, nivolumab, pembrolizumab, CT-011, AMP-224, and MDX-1106.

As used herein, PD-1 inhibitors and PD-L1 inhibitors refer to a group of checkpoint inhibitors or immune checkpoint inhibitors useful in the treatment of cancer. PD1 and PD-L1 are both proteins present on the surface of cells. PD-1 and PD-L1 inhibitors act to inhibit the association of the programmed death-ligand (PD-L1) with its receptor, programmed cell death protein 1 (PD-1). Exemplary PD-1 and/or PD-L1 inhibitors include, but are not limited to Nivolumab (Opdivo), Pembrolizumab (MK-3475 or lambrolizumab, Keytruda), Atezolizumab (Tecentriq), Avelumab (Bavencio), Durvalumab (Imfinzi), Tislelizumab, pidilizumab, AMP-224, AMP-514, PDR001, cemiplimab, BMS-936559, and CK-301.

In some embodiments, the PD-1 or PD-L1 inhibitor is Nivolumab (Opdivo), Pembrolizumab (MK-3475 or lambrolizumab, Keytruda), Atezolizumab (Tecentriq), Avelumab (Bavencio), Durvalumab (Imfmzi), or Tislelizumab.

Anti-PD-L1 antibodies and methods of making the same are known in the art. Such antibodies to PD-L1 may be polyclonal or monoclonal, and/or recombinant, and/or humanized. Exemplary PD-L1 antibodies are disclosed in U.S. Pat. Nos. 8,217,149, 8,383,796, 8,552,154, 9,212,224, and 8,779,108, and US Patent Appln. Pub. Nos. 20110280877, 20140341902, and 20130045201. Additional exemplary antibodies to PD-L1 (also referred to as CD274 or B7-H1) and methods for use are disclosed in U.S. Pat. Nos. 7,943,743, 8,168,179, and 7,595,048; WO2014055897, WO2016007235; and US Patent Appln. Pub. Nos. 20130034559 and 20150274835. In one embodiment, the anti-PD-L1 antibody is BMS-936559 (MDX-1105), MPDL3280A (RG7446), MED14736, TECENTRIQ™ (atezolizumab), YW243.55.S70, MPDL3280A, BMS-936559, MEDI4736, or MSB0010718C, or an antibody that comprises the VH and VL described in WO2013019906 (e.g., SEQ ID NOs: 21 and 24 therein). Examples of anti-PD-L1 antibodies and methods for making thereof are also described in WO 2010077634, WO 2007005874, WO 2011066389, WO 2013019906, WO 2010077634, U.S. Pat. Nos. 8,217,149 and 8,383,796, and US Patent Appln. Pub. No. 2013034559. PD-1 antagonists or PD-1 inhibitors refer to any chemical compound or biological molecule that blocks binding of PD-L1 expressed on a cancer cell to PD-1 expressed on an immune cell (T cell, B cell or NKT cell) and preferably also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCDIL1, PDL1, B7H1, B7-4, CD274 and B7-H for PD-Li; and PDCD1L2, PDL2, B7-DC, Btdc and CD273 for PD-L2. Human PD-1 amino acid sequences can be found in NCBI Locus No.: NP_005009. Human PD-L1 and PD-L2 amino acid sequences can be found in NCBI Locus No.: NP_054862 and NP_079515, respectively.

PD-1 antagonists can include, but are not limited to, a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. In some embodiments, the human constant region is selected from the group consisting of IgG1, IgG2, IgG3 and IgG4 constant regions, and in some embodiments, the human constant region is an IgG1 or IgG4 constant region. In some embodiments, the antigen binding fragment is selected from the group consisting of Fab, Fab′-SH, F(ab′)2, scFv and Fv fragments.

Examples of mAbs that bind to human PD-1 are described in U.S. Pat. Nos. 7,488,802, 7,521,051, 8,008,449, 8,354,509, and 8,168,757, WO 2004004771, WO 2004072286, WO 2004056875, and US Patent Appln. Pub. No. 20110271358. In one embodiment, anti-human PD-1 mAbs useful as the PD-1 antagonists include: MK-3475, nivolumab, the humanized antibodies h409All, h409A16 and h409A17, which are described in WO 2008156712, and AMP-514.

Other PD-1 antagonists useful in any of the aspects and embodiments of the present application include an immunoadhesin that specifically binds to PD-1, and preferably specifically binds to human PD-1, e.g., a fusion protein containing the extracellular or PD-1 binding portion of PD-L1 or PD-L2 fused to a constant region such as an Fc region of an immunoglobulin molecule. Examples of immunoadhesion molecules that specifically bind to PD-1 are described in WO 2010027827 and WO 2011066342. In one embodiment, the PD-1 antagonists include AMP-224 (also known as B7-DCIg), which is a PD-L2-FC fusion protein and binds to human PD-1.

In one embodiment, the anti-PD-1 antibody is KEYTRUDA/pembrolizumab, disclosed in U.S. Pat. No. 8,168,757 or Opdivo/nivolumab (also known as BMS-936558, MDX-1106, and ONO-4538, disclosed in U.S. Pat. No. 8,008,449.

In one embodiment, the CTLA-4 antagonist is Yervoy (ipilimumab), described in U.S. Pat. Nos. 6,984,720 and 7,605,238.

Additional examples of other therapeutic agents (anti-neoplastic agent) for use in combination or co-administration with a compound of the present application, for example, a compound of Formula (I), include antibodies to ICOS. Agonist antibodies to ICOS or ICOS binding proteins are disclosed in WO 2012013004, WO 2014033327, WO 2016120789, US Patent Appln. Pub. Nos. 20160215059 and US20160304610, for example, SEQ ID NOs: 1-6 or a direct equivalent thereof disclosed in WO 2016120789, a VH domain comprising an amino acid sequence at least 90% identical to SEQ ID NO: 7 as disclosed in WO 2016120789, and/or a VL domain comprising an amino acid sequence at least 90% identical to SEQ ID NO: 8 as disclosed in WO 2016120789.

Additional examples of other therapeutic agents (anti-neoplastic agent) for use in combination or co-administration with a compound of the present application, for example, a compound of Formula (I), include antibodies to OX040. Such antibodies are described in WO 2012027328, WO 2013028231 (e.g., an antibody comprising a VL having a sequence at least 90% identical to SEQ ID NO: 10 and/or a VH having a sequence at least 90% identical to SEQ ID NO:4 therein)

Additional examples of other therapeutic agents for use in combination or co-administered with a compound of the present application, for example, a compound of Formula (I), include immunostimulatory agents. As used herein immunostimulatory agent refers to any agent that can stimulate the immune system. As used herein immunostimulatory agents include, but are not limited to, vaccine adjuvants, such as Toll-like receptor agonists, T-cell checkpoint blockers, such as mAbs to PD-1 and CTL4 and T-cell checkpoint agonist, such as agonist mAbs to OX-40 and ICOS.

In one embodiment, TLR agonists include, but are not limited to, Pam3Cys, a TLRI/2 agonist; CFA, a TLR2 agonist; MALP2, a TLR2 agonist; Pam2Cys, a TLR2 agonist; FSL-I, a TLR-2 agonist; Hib-OMPC, a TLR-2 agonist; polyinosinic:polycytidylic acid (Poly IC), a TLR3 agonist; polyadenosine-polyuridylic acid (poly AU), a TLR3 agonist; Polyinosinic-Polycytidylic acid stabilized with poly-L-lysine and carboxymethylcellulose (Hiltonol), a TLR3 agonist; bacterial flagellin, a TLR5 agonist; imiquimod, a TLR7 agonist; resiquimod, a TLR7/8 agonist; loxoribine, a TLR7/8 agonist; and unmethylated CpG dinucleotide (CpG-ODN), a TLR9 agonist. Additional TLR agonists include, but are not limited to aminoalkyl glucosaminide phosphates (AGPs). An example of a naturally occurring TLR4 agonist is bacterial LPS. An example of a semisynthetic TLR4 agonist is monophosphoryl lipid A (MPL). Additional AGP derivatives are disclosed in U.S. Pat. Nos. 7,129,219, 6,525,028, and 6,911,434.

In one embodiment, the immunostimulatory agent for use in combination with the compounds of the present application is, for example, a compound of Formula (I), is a TLR4 agonist. In one embodiment, the TLR4 agonist is CRX-601, CRX-527, or CRX 547. Other embodiments include AGPs such as CRX 602 or CRX 526.

In addition, the compounds of the present application, for example, a compound of Formula (I), may be combined with other therapeutic agents which, because of their adjuvant nature, can act to stimulate the immune system to respond to the cancer antigens present on the inactivated tumor cell(s). Such adjuvants include, but are not limited to, lipids, liposomes, inactivated bacteria which induce innate immunity (e.g., inactivated or attenuated Listeriamonocytogenes), compositions which mediate innate immune activation via, (NOD)—like receptors (NLRs), Retinoic acid inducible gene-based (RIG)-I-like receptors (RLRs), and/or C-type lectin receptors (CLRs). Examples of PAMPs include lipoproteins, lipopolypeptides, peptidoglycans, zymosan, lipopolysaccharide, neisserial porins, flagellin, profillin, galactoceramide, muramyl dipeptide. Peptidoglycans, lipoproteins, and lipoteichoic acids are cell wall components of Gram-positive. Lipopolysaccharides are expressed by most bacteria, with MPL being one example. Flagellin refers to the structural component of bacterial flagella that is secreted by pathogenic and commensal bacteria, rt-Galactosylceramide (rt.-GalCer) is an activator of natural killer T (NKT) cells. Muramyl dipeptide is a bioactive peptidoglycan motif common to all bacteria.

Additional examples of other therapeutic agents for use in combination or co-administered with a compound of the present application, for example, a compound of Formula (I), include IDO inhibitors (e.g., Epacadostat, as disclosed in U.S. Pat. No. 8,034,953), CD73 inhibitors, and A2a and A2b adenosine antagonists.

In one embodiment, the compounds may be administered in combination with one or more separate pharmaceutical agents, e.g., a chemotherapeutic agent, an immunotherapeutic agent, or an adjunctive therapeutic agent.

The compounds of the present application, for example, a compound of Formula (I), may be used in combination with at least one other therapeutic agent useful in the prevention or treatment of bacterial and viral infections. Examples of such agents include, without limitation: polymerase inhibitors such as those disclosed in WO 2004037818 and WO 2006045613; JTK-003, JTK-019, NM-283, HCV-796, R-803, R1728, R1626, as well as those disclosed in WO 2006018725, WO 2004074270, WO 2003095441, US Appl. Pub. No. 20050176701, WO 2006020082, WO 2005080388, WO 2004064925, WO 2004065367, WO 2003007945, WO 2002004425, WO 2005014543, WO 2003000254, EP 1065213, WO 2001047883, WO 2002057287, WO 2002057245; replication inhibitors such as acyclovir, famciclovir, ganciclovir, cidofovir, lamivudine, and similar agents; protease inhibitors such as the HIV protease inhibitors saquinavir, ritonavir, indinavir, nelfinavir, amprenavir, fosamprenavir, brecanavir, atazanavir, tipranavir, palinavir, lasinavir, and the HCV protease inhibitors BILN2061, VX-950, SCH503034, and similar agents; nucleoside and nucleotide reverse transcriptase inhibitors such as zidovudine, didanosine, lamivudine, zalcitabine, abacavir, stavudine, adefovir, adefovir dipivoxil, fozivudine, todoxil, emtricitabine, alovudine, amdoxovir, elvucitabine, tenofovir disproxil fumarate, tenofovir alafenamide fumarate/hemifumarate, and similar agents; non-nucleoside reverse transcriptase inhibitors (including an agent having anti-oxidation activity such as immunocal, oltipraz etc.) such as nevirapine, delavirdine, efavirenz, loviride, immunocal, oltipraz, capravirine, TMC-278, TMC-125, etravirine, rilpivirine, and similar agents; entry inhibitors such as enfuvirtide (T-20), T-1249, PRO-542, PRO-140, TNX-355, BMS-806, 5-Helix, and similar agents; integrase inhibitors such as dolutegravir, elvitegravir, raltegravir L-870,180, and similar agents; budding inhibitors such as PA-344 and PA-457, and similar agents; chemokine receptor inhibitors such as vicriviroc (Sch-C), Sch-D, TAK779, maraviroc (UK-427,857), TAK449, as well as those disclosed in WO 2002074769, WO 20040054974, WO 2004055012, WO 2004055010, WO 2004055016, WO 2004055011, and WO 2004054581, and similar agents; pharmacokinetic enhancers such as cobicistat; neuraminidase inhibitors such as CS-8958, zanamivir, oseltamivir, peramivir, and similar agents; ion channel blockers such as amantadine or rimantadine, and similar agents; interfering RNA and antisense oligonucleotides and such as ISIS-14803 and similar agents; and antiviral agents of undetermined mechanism of action, for example those disclosed in WO 2005105761, WO 2003085375, WO 2006122011, ribavirin, and similar agents.

The compounds of the present application, for example, a compound of Formula (I), may be used in combination with at least one other therapeutic agent which may be useful in the treatment of Kaposi's sarcoma-associated herpesvirus infections (KSHV and KSHV-related) including, without limitation, chemotherapeutic agents such as bleomycin, vinblastine, vincristine, cyclophosphamide, prednisone, alitretinoin and liposomal anthracyclines such as doxorubicin, daunorubicin, immunotherapeutics such as Rituximab, Tocilizumab, Siltuximab, and others such as Paclitaxel and Rapamycin.

The compounds of the present application, for example, a compound of Formula (I), may be used in combination with at least one other therapeutic agent which may be useful in the treatment of TB infection Mycobacterium tuberculosis) and Tularemia (Franciseia tularensis), including, without limitation, first line oral agents isoniazid, Rifampicin, pyrazinamide, ethambutol, streptomycin, rifabutin; injectable agents including kanamycin, amikacin, capreomycin, streptomycin; fluoroquinolones including levofloxacin moxifloxacin ofloxacin; oral bacteriostatic agents para-aminosalicylic acid cycloserine terizidone thionamide protionamide; SQ-109 PNU-100480, Rifapentine Linezolid, PA-824 AZD5847, Gatifloxacin Moxifloxacin, Sirturo (bedaquiline) Delamanid (OPC-67683) and agents with undetermined mechanism of action in the treatment of drug-resistant TB, including clofazimine, linezolid, amoxicillin/clavulanate thioacetazone imipenem/cilastatin high dose isoniazid clarithromycin, and ciprofloxacin.

The compounds of the present application, for example, a compound of Formula (I), may be used in combination with at least one antimycobacterial agent (such as isoniazid (INH), ehambutol (Myambutol®), rifampin (Rifadin®), and pyrazinamide (PZA)), a bactericidal antibiotic (such as rifabutin (Mycobutin®) or rifapentine (Priftin®)), an aminoglycoside (Capreomycin®), a fluorquinolone (levofloxacin, moxifloxicin, ofloxacin), thioamide (ehionamide), cyclosporine (Sandimmune®), para-aminosalicyclic acid (Paser®),cycloserine (Seromycin®), kanamycin (Kantrex®), streptomycin, viomycin, capreomycin (Capastat®)), bedaquiline fumarate (Sirturo®), oxazolidinone (Sutezolid®), PNU-100480, or delamanid (OPC-67683).

The compounds of the present application, for example, a compound of Formula (I), may be used in combination with at least one other therapeutic agent which may be useful in the treatment of Chlamydia including, without limitation, Azithromycin, Doxycycline, Erythromycin, Levofloxacin, and Ofloxacin.

The compounds of the present application, for example, a compound of Formula (I), may be used in combination with at least one other therapeutic agent which may be useful in the treatment of Plasmodium infection including, without limitation, chloroquine, atovaquone-proguanil, artemether-lumefantrine, mefloquine, quinine, quinidine, doxocycline, cindamycin, artesunate, and primaquine.

The compounds of the present application, for example, a compound of Formula (I), may be used, for example in the treatment of amyotrophic lateral sclerosis (ALS), in combination with a glutamate blocker (Riluzole (Rilutek®)), quinidine (Nuedexta®), anticholinergics (amitriptyline®, Artane®, scopolamine patch (Transderm Scop®)), sympathomimetics (pseudoephedrine), mucolytics (guaifenesin), or analgesics (tramadol (Ultram®); ketorolac (Toradol®); morphine; or fentanyl patch (Duragesic®)).

The compounds of the present application, for example, a compound of Formula (I), may be used, for example in the treatment of multiple scelrosis, in combination with corticosteroids (prednisone, methylprednisolone), Interferon Beta 1-A (Avonex®, Extavia®, Rebif®, Betaseron®), peginterferon beta-IA (Plegridy®), Glatiramer acetate (Copaxone®); glatiramer acetate (Glatopa®-generic equivalent of Copaxone); Dimethyl fumarate (Tecfidera®); Fingolimod (Gilenya®)); teriflunomide (Aubagio®); dalfampridine (Ampyra®); daclizumab (Zinbryta); alemtuzumab (Lemtrada®); natalizumab (Tysabri®); or mitoxantrone hydrochloride (Novantrone®).

The compounds of the present application, for example, a compound of Formula (I) may be used in combination with one or more vaccines or immunogenic antigens useful in the prevention or treatment of viral infections. Such vaccines or immunogenic antigens include, without limitation, pathogen derived proteins or particles such as attenuated viruses, virus particles, and viral proteins typically used as immunogenic substances. Examples of viruses and viral antigens include, without limitation, Polioviruses, Coronaviridae and Coronaviruses, Rhinovirus (all subtypes), Adenoviruses (all subtypes), Hepatitis A, Hepatitis B, Hepatitis C, Hepatitis D, Human papillomavirus (including all subtypes), Rabies viruses, Human T-cell lympotropic virus (all subtypes), Rubella virus, Mumps virus, Coxsackie virus A (all subtypes), Cosackie virus B (all subtypes), human enteroviruses, herpesviruses including cytomegalovirus, Epstein-Barr virus, human herepes viruses (all subtypes), herpes simplex virus. varicella zoster virus, human immunodeficiency virus (HIV) (all subtypes), Epstein-Barr virus, Reoviruses (all subtypes), Filoviruses including Marburg virus and Ebola virus (all stains), Arenaviruses including Lymphocytic choriomeningitis virus, Lassa virus, Junin virus, and Machupo virus, Arboviruses including West Nile virus, Dengue viruses (all serotypes), Zika virus, Colorado tick fever virus. Sindbis virus, Togaviraidae, Flaviviridae, Bunyaviridae, Reoviridae, Rhabdoviridae, Orthomyxoviridae, Poxviruses including orthopoxvirus (variola virus, monkypox virus, vaccinia virus, cowpox virus), yatapoxviruses (tanapox virus, Yaba monkey tumor virus), parapoxvirus, molluscipoxvirus, Yellow fever, Hantaviruses including Hantaan, Seoul, Dobrava, Sin Nombre, Puumala, and Dobrava-like Saaremaa, human para influenza viruses and influenza viruses (all types), HLN1 influenza and swine influenza viruses, respiratory syncytial virus (all subgroups), rotaviruses including human rotaviruses A-E, bovine rotavirus, rhesus monkey rotavirus, Polyomaviruses including simian virus 40, JC virus, BK virus, Coltiviruses, eyach virus, calciviruses, and Parvoviridae including dependovirus, parvovirus and erythrovirus.

The compounds of the present application, for example, a compound of Formula (I), may be used in combination with at least one other therapeutic agent which may be useful in the prevention or treatment of viral infections for example immune therapies (e.g., interferon or other cytokines/chemokines, cytokine/chemokine receptor modulators, cytokine agonists or antagonists and similar agents); and therapeutic vaccines, anti-fibrotic agents, anti-inflammatory agents such as corticosteroids or NSAIDs (non-steroidal anti-inflammatory agents) and similar agents.

The compounds of the present application, for example, a compound of Formula (1), may be used in combination with other anti-inflammatory agents, including oral or topical corticosteroids, anti-TNF agents, 5-aminosalicyclic acid and mesalamine preparations, hydroxycloroquine, thiopurines, methotrexate, cyclophosphamide, cyclosporine, calcineurin inhibitors, mycophenolic acid, mTOR inhibitors, JAK inhibitors, Syk inhibitors, anti-inflammatory biologic agents, including anti-IL6 biologies, anti-ILl agents, anti-IL17 biologies, anti-CD22, anti-integrin agents, anti-IFNa, anti-CD20 or CD4 biologies and other cytokine inhibitors or biologies to T-cell or B-cell receptors or interleukins.

The compounds of the present application, for example, a compound of Formula (I), may be used in combination, for example in the treatment of systemic lupus erythematosus and related lupus disorders, with at least one other therapeutic agent, including, but not limited to, a corticosteroid (such as prednisolone (Delatsone®, Orapred, Millipred, Omnipred, Econopred, Flo-Pred), an immunosuppressive agent (such as methotrexate (Rhuematrex®, Trexall®), dexamethasone (DecadronR, Solurex®), Mycophenolate mofetil (Cellcept®), Tacrolimus®, Sirolimus®), B-cell therapy (belimumab (Benlysta®), B-cell inhibitor (Atacicept®, Apratuzumab® (anti-CD22), SBI-087 (anti-CD20), an anti-BAFF antibody (LY2127399, A623), Velcade®), azathioprine (Azasan®, Imuran®), triamcinolone (Clinacort®, Kenalog-10®), hydroxychloroquine (Plaquenil®), thalidomide (Immunoprin®, Contergan®), immunoglobulin therapy (HyQiva®, Flebogamma®, Gamunex®, Privigen®, Gammagard®), anti-interferon-alpha therapy (Rontalizumab®, Sifalimumab®, AGS-009R, IFN Kinoid), TLR7 and TLR9 blockers (IMO-3100), anti-cytokine therapies (anti-IL6 (CNTO-136), anti-interferon-gamma (AMG811), immunomodulatory therapy (Lupuzor™, Abatacept, Orencia®, AMG557, Laquinimod, Paquinimod, Leflunomide, anti-ICOS (Medi-570), anti-CD40 ligand antibody (CDP7657)), and/or a platelet aggregation inhibitor (aspirin).

The compounds of the present application, for example, a compound of Formula (I), may be used, for example in the treatment of vasculitis and disease with inflammation of small or medium size blood vessels, in combination with alkylating agents (cyclophosphamide, Cytoxan®), anti-rheumatic anti-CD20 antibody (Rituxan®, Rituximab®), and anti-TNF inhibitors (Etanrcept®).

The compounds of the present application, for example, a compound of Formula (I) may be used, for example in the treatment of psoriasis, in combination with ixekizumab, tildrakizumab (MK-3222), or secukinumab (AlN457).

A second agent can be selected from an inhaled corticosteroid, a long-acting beta agonist, a combination of an inhaled corticosteroid and a long-acting beta agonist, a short acting beta agonist, a leukotriene modifier, an anti-IgE, a methylxanthine bronchodilator, a mast cell inhibitor, and a long-acting muscarinic antagonist. The compounds of the present application may be used, for example in the treatment of asthma, in combination with an inhaled corticosteroid ((ICS) such as fluticasone proprionate (Flovent®), beclomethasone dipropionate (QVAR®), budesonide (Pulmicort), trimcinolone acetonide (Azmacortk), flunisolide (Aerobid®), mometasone fuorate (Asmanex0 TwisthalerO), or Ciclesonide (Alvesco®)), a long acting beta agonist ((LABA) such as formoterol fumarate (Foradil®), salmeterol xinafoate (Serevent®)), a combination of an ICS and LABA (such as fluticasone furoate and vilanterol (Breo ElliptaR), formoterol/budesonide inhalation (Symbicort®), beclomethasone dipropionate/formoterol (Inuvair®), and fluticasone propionate/salmeterol (Advair®), a short acting beta agonist ((SABA) such as albuterol sulfate (ProAir®, Proventil HFA®, Ventolin HFA®. AccuNeb® Inhalation Solution), levalbuterol tartrate (Xopenex® HFA), ipratropium bromide/albuterol (Combivent® Respimat®), ipratropium bromide (Atrovent® HFA), a leukotriene modifier (such as montelukast sodium (Singulair®), zafirlukast (Accolate®),or zileuton (Zyflo®), and anti-IgE (such as omalizumab (Xolair®)), a methylxanthine bronchodilator (such as theophylline (Accurbron®, Aerolate®, Aquaphyllin®, Asbron®, Bronkodyl®, Duraphyl®, Elixicon®, Elixomin®, Elixophyllin®, Labid®, Lanophyllin®, Quibron-T®, Slo-Bid®, Slo-Phyllin®, Somophyllin®, Sustaire®, Synophylate®, T-Phyll®, Theo-24®, Theo-Dur®, Theobid®, Theochron®, Theoclear®, Theolair®, Theolixir®, Theophyl®, Theovent®, Uni-dur®, Uniphyl®), a mast cell inhibitor (such as cromulyn sodium (Nasalcrom®) and nedocromil sodium (Tilade®)), a long-acting muscarinic antagonist ((LAMA) such as mometasone furoate/formoterol fumarate dihydrate (Dulera®)).

Second agents can also include, but are not limited to, a protein tyrosine kinase inhibitor (masitinib), CRTH2/D-prostanoid receptor antangonist (AMG 853), indacaterol (Arcapta® Neohaler®), an epinephrine inhalation aerosol (E004), fluticasone furoate/fluticasone proprionate, vinanterol inhalation/fluticasone furoate powder (Relovair™), fluticasone propionate/eformoterol fumarate dehydrate (Flutiform®), reslizumab, salbutamol dry-powder inhalation, tiotropium bromide (Spiriva® HandiHaler®), fomloterol/budesonide (Symbicort® SMART®), fluticasone furoate (Veramyst®), Vectura's VR506, lebrikizumab (RG3637), a combination phosphodiesterase (PDE)-3 and (PDE)-4 inhibitor (RPL554).

Second agents can also include, but are not limited to, a long-acting beta agonist, a long-acting inhaled anticholinergic or muscarinic antagonist, a phosphodiesterase inhibitor, a combination an inhaled corticosteroid long-acting beta agonist, a short acting beta agonist, and an inhaled corticosteroid. For example, in the treatment of COPD, the compounds of the present application may be used in combination with a LABA (such as salmeterol xinafoate (Serevent), umeclidinium/vilanterol (Anoro Ellipta®), umeclidinium (Incruse ElliptaR), aformoterol tartrate (Brovana®), formoterol fumarate inhalation powder (Foradil®), indacterol maleate (ArcaptaR Neohaler®), or fluticasone propionate/eformoterol fumarate dehydrate (Flutiform®)), a long-acting inhaled anticholinergic (or muscarinic antagonist, such as tiotropium bromide (Spiriva®), and aclidinium bromide (Tudorza® Pressair®), a phosphodiesterase (PDE-r) inhibitor (such as roflumilast, Daliresp®), a combination ICS/LABA (such as fluticasone furoate and vilanterol (Breo Ellipta3), fluticasone propionate/salmeterol (Advair®), budesonide/formoterol (Symbicort®), mometasone/formoterol (Dulera®), ipratropium bromide/albuterol sulfate (Duoneb®, Atrovent®), albuterol/ipratropium (Combivent Respimat®)), a SABA (such as ipratropium bromide (Atrovent®), and albuterol sulfate(ProAir®,Proventil®)), and an ICS (such as budesonide (Pulmicort®) and fluticasone propionate (Flovent®), beclometasone dipropionate (QVAR®).

Other agents that may be suitable for use, for example in the treatment of COPD, in combination therapy with the compounds of the present disclosure, for example, a compound of Formula (I), include but are not limited to, SCH527123 (a CXCR2 antagonist), glycoprronium bromide ((NVA237) Seebri® Breezhaler®), glycopyrronium bromide and indacaterol maleate ((QVA149) Ultibro® Breezhaler®), glycopyrrolate and formoterol fumarate (PT003), indacaterol maleate (QVA149), olodaterol (Striverdi® Respimat®), tiotropium (Spiriva®)/olodaterol (Striverdi® Respimat®), and aclidinium/formoterol inhalation.

A second agent can include, but is not limited to, an oral corticosteroid, anti-thymocyte globulin, thalidomide, chlorambucil, a calcium channel blocker, a topical emollient, an ACE inhibitor, a serotonin reuptake inhibitor, an endothelin-1 receptor inhibitor, an anti-fibrotic agent, a proton-pump inhibitor or imatinib, ARG201, and tocilizumab.

The compounds of the present application, for example, a compound of Formula (I), may be used, for example in the treatment of systemic scleroderma, in combination with an oral corticosteroid (such as prednisolone (DelatsoneR, Orapred, Millipred, Onipred, Econopred, Flo-Pred), an immunosuppressive agent (such as methotrexate (Rhuematrex®, Trexall®), cyclosporine (Sandimmune®), anti-thymocyte globulin (Atgam®), mycophenolate mofetil (CellCept®), cyclophosphamide (Cytoxan®), FK506 (tacrolimus), thalidomide (Thalomid®), chlorambucil (Leukeran®), azathioprine (Imuran®, Azasan®)), a calcium channel blocker (such as nifedipine (Procardia®, Adalat®) or nicardipine (Cardene®), a topical emollient (nitroglycerin ointment), an ACE inhibitor (such as lisinopril (Zestril®, Prinivil®), diltaizem (Cardizem®, Cardizem SR®, Cardizem CD®, Cardiai, Dilacor®, Tiazac®)), a serotonin reuptake inhibitor (such as fluoxetine (Prozac®)), an endothelin-1 receptor inhibitor (such as bosentan (Tracleer®) or epoprostenol (Flolan®, Veletri®, Prostacyclin®)) an anti-fibrotic agent (such as colchicines (Colcrys®), para-aminobenzoic acid (PABA), dimethyl sulfoxide (DMSO), and D-penicillamine (Cuprimine®, DepenCR®), interferon alpha and interferon gamma (INF-g)), a proton-pump Inhibitor (such as omeprazole (Prilosec®), metoclopramide (Reglan®), lansoprazole (Prevacid®), esomeprazole (Nexium®), pantoprazole (Protonix®), rabeprazole (Aciphex®)) or imatinib (Gleevec®) ARG201 (arGentis Pharmaceutical), belimumab (Benlysta®), tocilizumab (Actema®).

The compounds of the present application, for example, a compound of Formula (I), may be used, for example in the treatment of Sjogren's syndrome, in combination with anti-rheumatic agents (hydroxychloroquine and Plaquenil®, Ridaura®, Kineret®), cholinergic agonists (Salagen®, Evoxac®), a JAK inhibitor (Xelijanz®, and anti-TNF treatments (Remicade®, Humira®, Enbrel®, Cimzia®, Simponi®).

A second agent can include, but is not limited to, a ciliary neurotrophic growth factor or a gene transfer agent. For example, in the treatment of retinitis pigmentosa, the compounds of the present application may be used in combination with a ciliary neurotrophic growth factor (NT-501-CNTF) or gene transfer agent, UshStat®.

A second agent can include, but is not limited to, a trivalent (IIV3) inactivated influenza vaccine, a quadrivalent (IIV4) inactivated influenza vaccine, a trivalent recombinant influenza vaccine, a quadrivalent live attenuated influenza vaccine, an antiviral agent, or inactivated influenza vaccine. For example, in the treatment of influenza, the compounds of the present application, for example, a compound of Formula (I), may be used in combination with a trivalent (IIV3) inactivated influenza vaccine (such as Afluria®, Fluarix®, Flucelvax®, FluLaval®, Fluvirin®, Fluzone®), a quadrivalent (IIV4) inactivated influenza vaccine (such as Fluarix® Quadrivalent, Flulaval® Quadrivalent, Fluzone® Quadrivalent), a trivalent recombinant influenza vaccine (such as FluBlok®), a quadrivalent live attenuated influenza vaccine (such as FluMist® Quadrivalent), an antiviral agent (such as oseltamivir (Tamiflu®), zanamivir (Relenza®), rimantadine (Flumadine®), or amantadine (Symmetrel®)), or Fluad®, Fludase, FluNhance®, Preflucel, or VaxiGrip®.

The compounds of the present application, for example, a compound of Formula (1), may be used, for example in the treatment of a staphylococcus infection, in combination with an antibiotic (such as a B-Lactam cephalosporin (Duricef®, Kefzol®, AncefC, BiocefR, etc), nafcillin (Unipen®), a sulfonamide (sulfamethoxazole and trimethoprim (Bacrim®, Septra®,) sulfasalazine (Azulfidine®), acetyl sulfisoxazole (Gantrisin®), etc), or vancomycin (Vancocin®)).

A second agent can include, but is not limited to, a topical immunomodulator or calcineurin inhibitor, a topical corticosteroid, an oral corticosteroid, an interferon gamma, an antihistamine, or an antibiotic. The compounds of the present application, for example, a compound of Formula (I), may be used, for example in the treatment of atopic dermatitis, in combination with a topical immunomodulator or calcineurin inhibitor (such as pimecrolimus (Elidel®) or tacrolimus ointment (Protopic®)), a topical corticosteroid (such as hydrocortizone (Synacort®, Westcort®), betamethasone (DiproleneR), flurandrenolide (Cordan®), fluticasone (Cutivate®), triamcinolone (Kenalog®), fluocinonide (Lidex®), and clobetasol (Temovate®)), an oral corticosteroid (such as hydrocortisone (Cortef®), methyl prednisolone (Medrol®), or prednisolone (Pediapred®, Prelone®), an immunosuppressant (such as cyclosporine (Neoral®) or interferon gamma (Alferon N®, Infergen®, Intron A, Roferon-A®)), an antihistamine (for itching such as Atarax®, Vistaril®, Benadryl®), an antibiotic (such as penicillin derivatives flucloxacillin (Floxapen®) or dicloxacillin (Dynapen®), erythromycin (Eryc®, T-Stat®, Erythra-Derm®, etc.)), a non-steroidal immunosuppressive agent (such as azathioprine (Imuran®, Azasan®), methotrexate (Rhuematrex®, Trexall®), cyclosporin (Sandimmune®), or mycophenolate mofetil (CellCept®)).

In another aspect, a second agent can include, but is not limited to a cytokine such as G-CSF (granulocyte colony stimulating factor). In another aspect, a compound of the present invention, for example, a compound of Formula (I), or a pharmaceutically acceptable salt, prodrug, metabolite, analog or derivative thereof, may be administered in combination with radiation therapy. Radiation therapy can also be administered in combination with a compound of the present invention, for example, a compound of Formula (I), and another chemotherapeutic agent described herein as part of a multiple agent therapy. In yet another aspect, a compound of the present invention, for example, a compound of Formula (I), or a pharmaceutically acceptable salt, prodrug, metabolite, analog or derivative thereof, may be administered in combination with standard chemotherapy combinations such as, but not restricted to, CMF (cyclophosphamide, methotrexate and 5-fluorouracil), CAF (cyclophosphamide, adriamycin and 5-fluorouracil), AC (adriamycin and cyclophosphamide), FEC (5-fluorouracil, epirubicin, and cyclophosphamide), ACT or ATC (adriamycin, cyclophosphamide, and paclitaxel), rituximab, Xeloda (capecitabine), Cisplatin (CDDP), Carboplatin, TS-1 (tegafur, gimestat and otastat potassium at a molar ratio of 1:0.4:1), Camptothecin-11 (CPT-11, Irinotecan or Camptosar™) or CMFP (cyclophosphamide, methotrexate, 5-fluorouracil and prednisone).

In some embodiments, a compound of the present invention, for example, a compound of Formula (I), or a pharmaceutically acceptable salt, prodrug, metabolite, polymorph or solvate thereof, may be administered with an inhibitor of an enzyme, such as a receptor or non-receptor kinase. Receptor and non-receptor kinases of the invention are, for example, tyrosine kinases or serine/threonine kinases. Kinase inhibitors of the invention are small molecules, polynucleic acids, polypeptides, or antibodies.

### Pharmaceutical Compositions

The present application also provides pharmaceutical compositions comprising the compound of Formula (I), or a pharmaceutically acceptable salt thereof, in combination with at least one pharmaceutically acceptable excipient or carrier.

A “pharmaceutical composition” is a formulation containing the compound of the present application in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or a pharmaceutically acceptable salt, tautomer, prodrug, solvate, metabolite, polymorph, analog or derivative thereof thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration.

A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this application include powders, sprays, ointments, pastes, creams, lotions, gels, solutions, patches and inhalants. In one embodiment, the active compound is mixed under sterile conditions with a pharmaceutically acceptable carrier, and with any preservatives, buffers or propellants that are required.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

A pharmaceutical composition of the application is formulated to be compatible with its intended route of administration. Examples of routes of administration include parenteral, e.g., intravenous. intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A compound or pharmaceutical composition of the application can be administered to a subject in many of the well-known methods currently used for chemotherapeutic treatment. For example, for treatment of cancers, a compound of the application may be injected directly into tumors, injected into the blood stream or body cavities or taken orally or applied through the skin with patches. The dose chosen should be sufficient to constitute effective treatment but not as high as to cause unacceptable side effects. The state of the disease condition (e.g., cancer, precancer, and the like) and the health of the patient should preferably be closely monitored during and for a reasonable period after treatment.

The term “therapeutically effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

For any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compound (i.e., the compound of Formula (I)) of the present application may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compound into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compound is delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compound is formulated into ointments, salves, gels, or creams as generally known in the art.

The active compound can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the application is dictated by and directly dependent on the unique characteristics of the active compound and the particular therapeutic effect to be achieved.

In therapeutic applications, the dosages of the pharmaceutical compositions used in accordance with the application vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and also preferably causing complete regression of the cancer. Dosages can range from about 0.01 mg/kg per day to about 5000 mg/kg per day. An effective amount of a pharmaceutical agent is that which provides an objectively identifiable improvement as noted by the clinician or other qualified observer. For example, regression of a tumor in a patient may be measured with reference to the diameter of a tumor. Decrease in the diameter of a tumor indicates regression. Regression is also indicated by failure of tumors to reoccur after treatment has stopped. As used herein, the term “dosage effective manner” refers to amount of an active compound to produce the desired biological effect in a subject or cell.

The pharmaceutical compositions can be included in a container, pack, or dispenser together with instructions for administration.

It should be understood that all references to pharmaceutically acceptable salts include solvent addition forms (solvates) or crystal forms (polymorphs) as defined herein, of the same salt.

The compound of the present application can also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., an acetate, propionate or other ester.

The compound, or pharmaceutically acceptable salts thereof, are administered orally, nasally, transdermally, pulmonary, inhalationally, buccally, sublingually, intraperintoneally, subcutaneously, intramuscularly, intravenously, rectally, intrapleurally, intrathecally and parenterally. In one embodiment, the compound or a pharmaceutically 15 acceptable salt thereof is administered orally. One skilled in the art will recognize the advantages of certain routes of administration.

The dosage regimen utilizing the compound is selected in accordance with a variety of factors including type, species, age, weight, sex and medical condition of the patient; the severity of the condition to be treated; the route of administration; the renal and hepatic function of the patient; and the particular compound or pharmaceutically acceptable salt, tautomer, prodrug, solvate, metabolite, polymorph, analog or derivative thereof employed. An ordinarily skilled physician or veterinarian can readily determine and prescribe the effective amount of the drug required to prevent, counter or arrest the progress of the condition.

Techniques for formulation and administration of the disclosed compound of the application can be found in Remington: the Science and Practice ofPharmacy, 19^{th} edition, Mack Publishing Co., Easton, PA (1995). In an embodiment, the compound described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compound or pharmaceutically acceptable salts thereof will be present in such pharmaceutical compositions in amounts sufficient to provide the desired dosage amount in the range described herein.

All percentages and ratios used herein, unless otherwise indicated, are by weight. Other features and advantages of the present application are apparent from the different examples. The provided examples illustrate different components and methodology useful in practicing the present application. The examples do not limit the claimed application. Based on the present application the skilled artisan can identify and employ other components and methodology useful for practicing the present application.

### Examples

The application is further illustrated by the following examples and synthesis schemes, which are not to be construed as limiting this application in scope or spirit to the specific procedures herein described. It is to be understood that the examples are provided to illustrate certain embodiments and that no limitation to the scope of the application is intended thereby. It is to be further understood that resort may be had to various other embodiments, modifications, and equivalents thereof which may suggest themselves to those skilled in the art without departing from the spirit of the present application and/or scope of the appended claims.

### Studies on the Combination of Compound of Formula I and Venetoclax

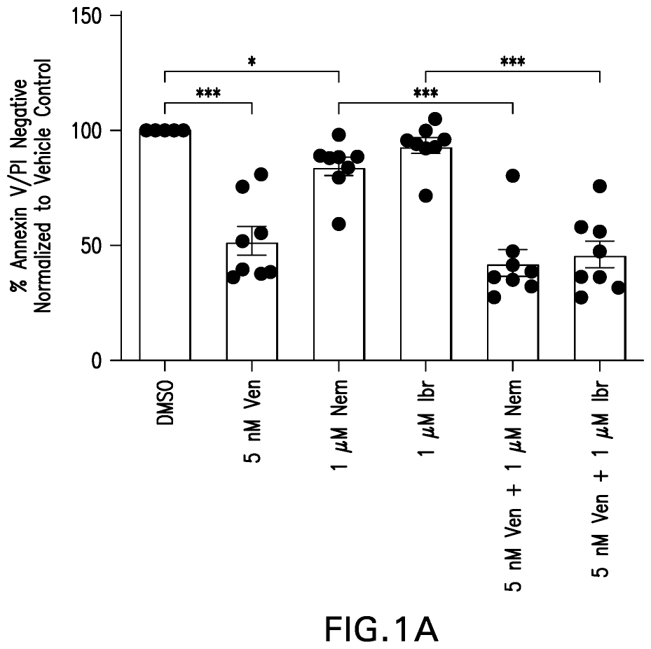

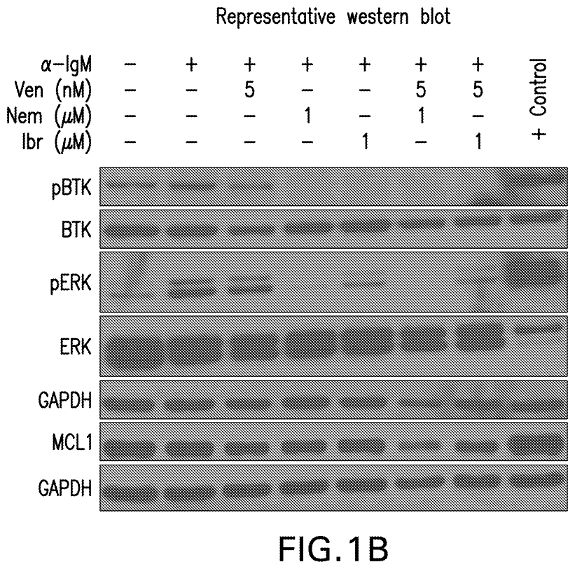

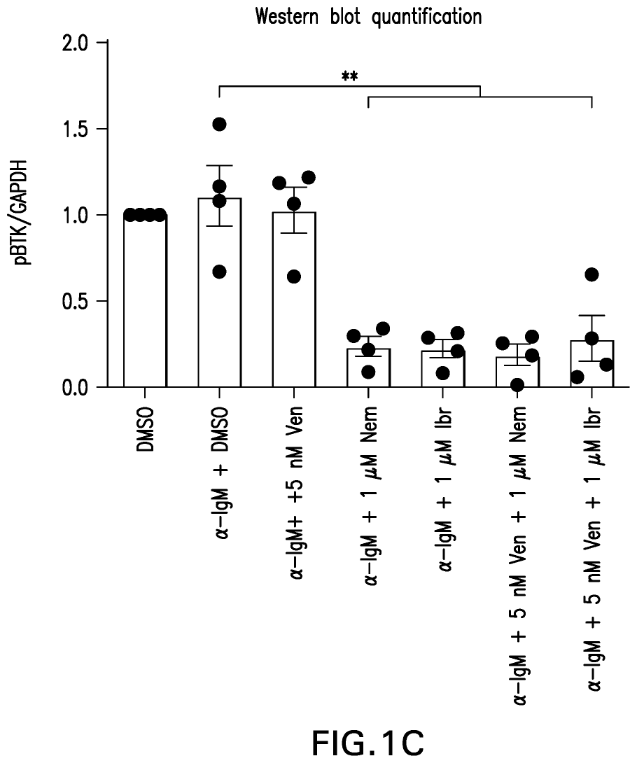

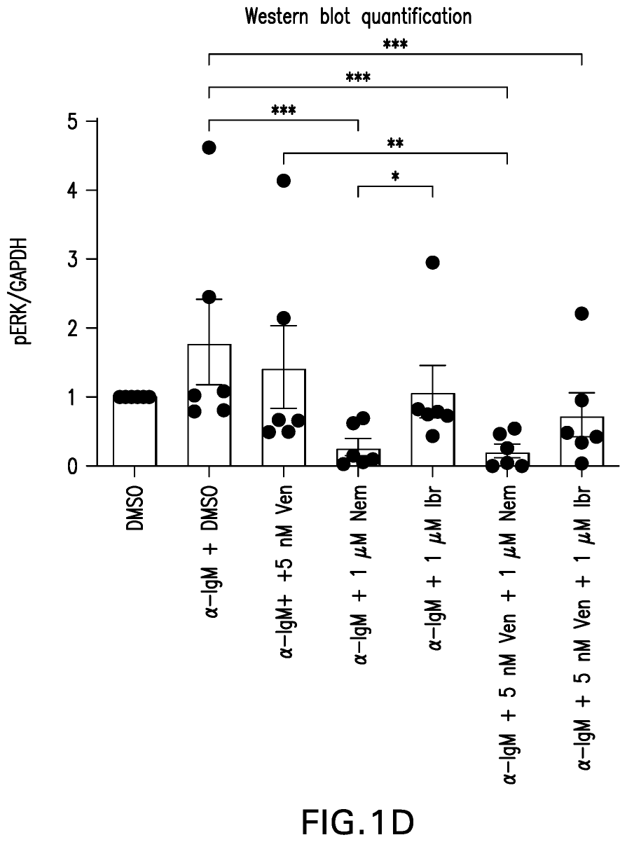

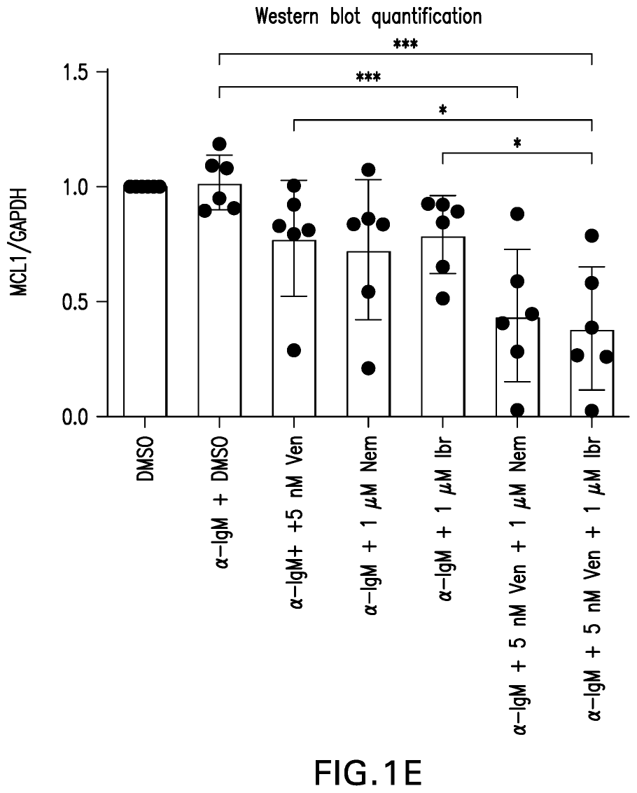

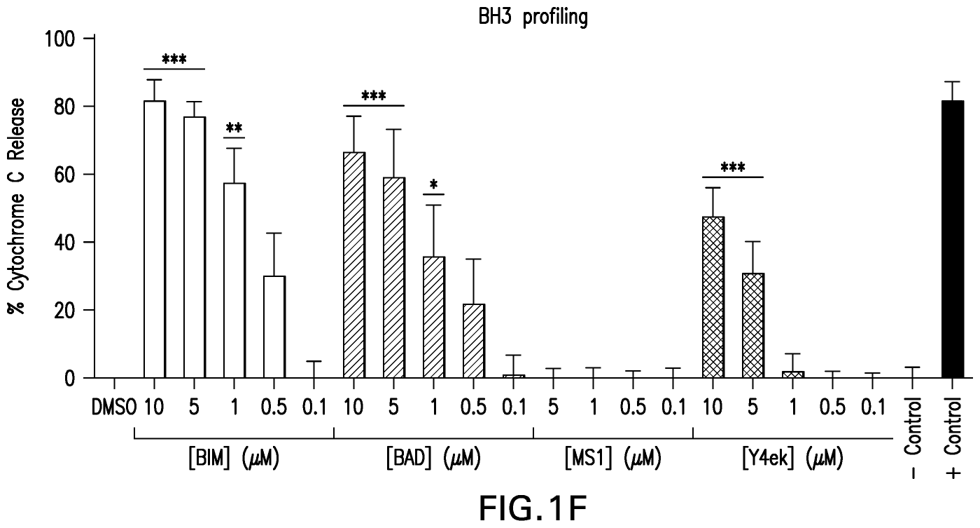

A study in primary CLL cells, demonstrated that there was a significantly decreased viability of cells treated with venetoclax compared to vehicle (p<0.0001); the addition of ibrutinib or nemtabrutinib increased cytotoxicity by 6 and 10%, respectively, compared to venetoclax alone (FIG. 1A). Primary CLL cells were treated with DMSO, venetoclax (5 nM), nemtabrutinib (1 μM), ibrutinib (1 μM) or in combination. Ibrutinib was washed out after 1 hr and total incubation time was 24 hr. Cell viability was assessed by flow cytometry following Annexin V/PI staining. Data are normalized to DMSO control. A linear mixed effect model was used to analyze raw data and p values were adjusted for multiple comparisons using Tukey's method (*, p<0.05, **, pS0.01, ***, pS0.001).

The effects of combining venetoclax and BTKi's on BCR signaling can be seen in FIG. 1B. Primary CLL cells were treated with DMSO, venetoclax (5 nM), nemtabrutinib (1 M), ibrutinib (1 μM) or in combination. Ibrutinib was washed out after 1 hr and total incubation time was 24 hr. Cells were stimulated with 10 μg plate-bound anti-IgM for 15 min before collection of whole cell lysate and analysis using SDS-PAGE.

For FIG. 1C to IE, all immunoblots of primary CLL patient samples were quantified using densitometry software (AlphaView). Protein levels (FIG. 1C: pBTK, FIG. 1D: pERK, FIG. 1E: MCL-1) normalized to GAPDH loading control are reported as fold change over vehicle control. A linear mixed effect model was used to analyze raw data normalized to GAPDH loading control and p values were adjusted for multiple comparisons using Tukey's method (*, p<0.05, ** p<0.01, ***, p<0.001).

Both ibrutinib and nemtabrutinib significantly inhibited autophosphorylation of BTK^{Y223} (ibrutinib vs stimulated vehicle p=0.0021, nemtabrutinib vs stimulated vehicle p=0.0024), which was unaffected by addition of venetoclax (FIG. 1C). ERK phosphorylation was inhibited to a more significant degree with nemtabrutinib than ibrutinib (nemtabrutinib vs stimulated vehicle p<0.0001, nemtabrutinib vs ibrutinib p=0.0461), and this inhibition was not affected by the addition of venetoclax (FIG. 1D). However, the BCL2 family protein MCL1 expression was significantly reduced by the combination of BTKi's plus venetoclax (ibrutinib combination vs stimulated vehicle p=0.0001, nemtabrutinib combination vs stimulated vehicle p=0.0003). Moreover, ibrutinib plus venetoclax significantly reduced MCL-1 expression compared to either as a single agent (combination vs ibrutinib p=0.0171, combination vs venetoclax p=0.0262) (FIG. 1E).

For FIG. 1F, primary CLL cells from nemtabrutinib treated patients were co-cultured with NK Tert stromal cells before performing BH3 profiling using BIM, BAD, MS-1, Y4ek, and PUMA2A peptides. Baseline cytochrome c release measured in DMSO treated cells has been subtracted from all test conditions and controls presented. Cytochrome c release in response to interaction with BIM, BAD, and Y4ek peptides indicates cellular dependency on BCL-2 and BCL-xL. A linear mixed effect model was used to analyze raw data and p values were adjusted for multiple comparisons using Tukey's method. All statistics represented are in comparison to DMSO treated control (*, p<0.05, **, p<0.01, ***, p<0.001). BH3 profiling of samples from 3 nemtabrutinib treated patients showed cytochrome c release in response to interaction with BIM, BAD, and Y4ek, indicating that these CLL cells maintain dependence on BCL2 and BCL-xL (FIG. 1F) (Ryan J, et al., Biol Chem. 2016; 397(7):671-8). This demonstrated venetoclax sensitivity.

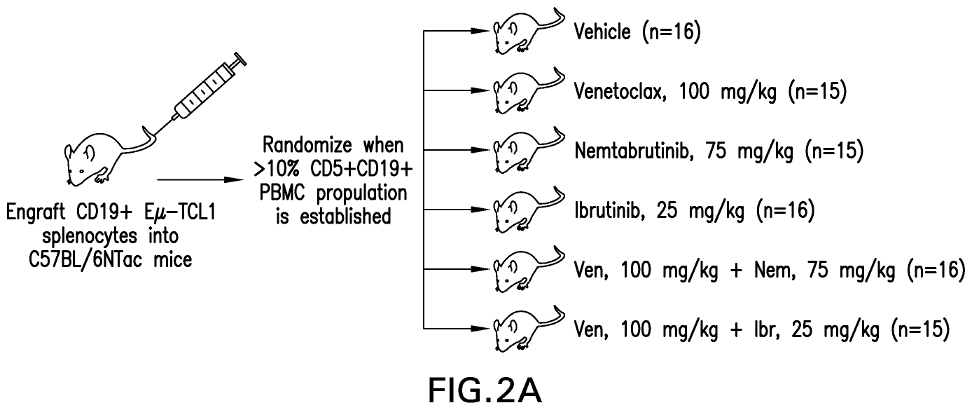

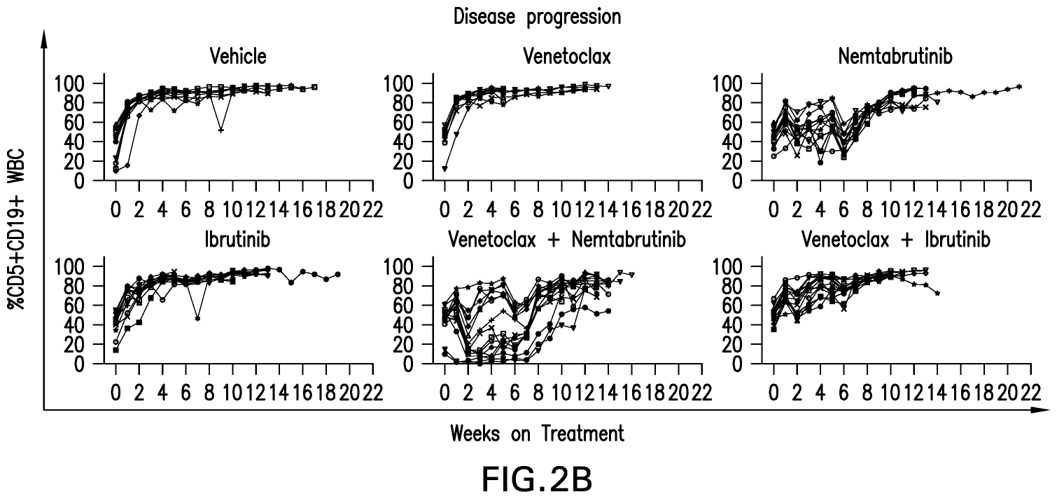

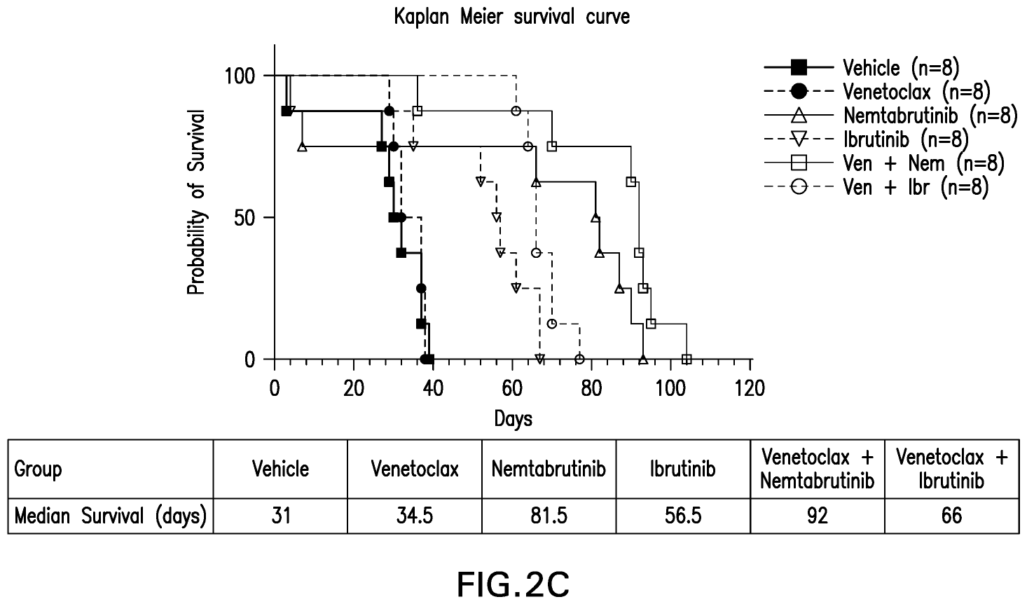

The Eμ-TCL1 adoptive transfer CLL mouse model was used to test the in vivo efficacy of venetoclax in combination with ibrutinib or nemtabrutinib. (Bichi R, et al. Proc Natl Acad Sci USA. 2002; 99(10):6955-60). For FIG. 2A, splenocytes from a Eμ-TCL1 donor mouse were engrafted into 93 C57BL/6NTac mice via tail vein injection. Recipient mice were randomly enrolled into treatment groups after developing CD5+CD19+ disease as assessed by flow cytometry of peripheral blood. Mice were treated daily via oral gavage with vehicle, venetoclax (100 mg/kg), nemtabrutinib (75 mg/kg), ibrutinib (25 mg/kg), venetoclax (100 mg/kg) and nemtabrutinib (75 mg/kg), or venetoclax (100 mg/kg) and ibrutinib (25 mg/kg). Due to the large study size, splenocytes from multiple E-TCL1 donors were needed to engraft C57BL/6NTac mice. Splenocytes from 4 donors were engrafted into groups of 48, 25, 17, and 3 recipient mice, respectively.

Peripheral blood disease progression was monitored weekly via flow cytometry and was reported as % CD5+CD19+ of CD45+ cells. (FIG. 2B). High variability was observed between recipient groups, including groups that did not have BCR-dependent disease as evidenced by lack of response to ibrutinib. These groups were excluded from analysis. The survival differences of 48 mice engrafted with splenocytes from a single donor with BCR-dependent disease were assessed. Ibrutinib treated mice reached a median survival of 56.5 days while mice treated with ibrutinib and venetoclax had a median survival of 66 days (ns, p=pending). Nemtabrutinib treated mice reached a median survival of 81.5 days while mice treated with nemtabrutinib and venetoclax reached a median survival of 92 days (preliminary p=0.03). Mice treated with vehicle and venetoclax had similar overall survival (median 31 days and 34.5 days, respectively). Furthermore, the combination of nemtabrutinib with venetoclax prolonged survival compared to the combination of ibrutinib and venetoclax (preliminary p=0.003).

FIG. 2C demonstrates Kaplan Meier estimated survival of mice with BCR-dependent disease treated with BTKi, venetoclax, or in combination. Inhibition of BCR signaling by nemtabrutinib is not affected by the addition of venetoclax, while the cytotoxicity of venetoclax is not affected by the addition of nemtabrutinib. The in vivo data showed that combinatorial treatment with nemtabrutinib and venetoclax is superior to treatment with either drug alone.

Additional studies of nemtabrutinib plus venetoclax versus venetoclax plus rituximab Objectives, and Endpoints

In participants aged at least 18 years with relapsed or refractory CLL/SLL who have failed at least 1 prior line of therapy:

| Objectives | Endpoints |
|---|---|
| Primary | |
| Objective (Part 1): To evaluate the | Dose Limiting Toxicities (DLT) |
| safety and tolerability and to | Adverse Events (AEs) |
| confirm the dose of nemtabrutinib | Study intervention discontinuations |
| in combination with venetoclax | due to AEs |
| Objective (Part 2): To compare | Progression-Free Survival (PFS), |
| nemtabrutinib + venetoclax to | defined as the time from random- |
| venetoclax + rituximab (VR) with | ization to the first documented |
| respect to PFS per 2018 iwCLL | disease progression or death due |
| criteria, as assessed by BICR | to any cause, whichever occurs |
| Secondary | first |
| Objective (Part 2): To compare | Undetectable MRD, defined as <1 |
| nemtabrutinib + venetoclax to VR | leukemic cell per 10,000 cells |
| with respect to undetectable | (MRD <10⁻⁴) in bone marrow |
| minimal residual disease (MRD) | |
| rate in bone marrow at month 14 | |
| as assessed by central laboratory | |
| Objective (Part 2): To compare | Overall Survival (OS), defined as |
| nemtabrutinib + venetoclax to VR | the time from randomization to |
| with respect to OS | death due to any cause |
| Objective (Part 2): To compare | OR, defined as Complete |
| nemtabrutinib + venetoclax to VR | Response/Remission (CR), Complete |
| with respect to ORR per iwCLL | Response with incomplete count |
| criteria 2018 as assessed by BICR | recovery (CRi), Nodular Partial |
| | Remission (nPR), or Partial |
| | Response/Remission (PR) |
| Objective (Part 2): To evaluate | DOR, defined as the time from the |
| Duration of Response (DOR) per | first documented evidence of CR, |
| 2018 iwCLL criteria as assessed | CRi, nPR, or PR that led to response |
| by BICR | until disease progression or death |
| | due to any cause, whichever occurs |
| | first |
| Objective (Part 2): To evaluate the | AEs |
| safety and tolerability of | Study intervention discontinuations |
| nemtabrutinib + venetoclax | due to AEs |
| Tertiary/Exploratory | |
| Objective (Part 2): To evaluate | OR including Partial Response with |
| nemtabrutinib + venetoclax | Lymphocytosis (PRL), defined |
| compared to VR with respect to | as CR, CRi, nPR, PR, or PRL |
| ORR, including PRL as a response | |
| category per 2018 iwCLL criteria | |
| as assessed by BICR | |
| Objective (Part 2): To characterize | Steady-state maximum concentration |
| the pharmacokinetics of | (Cₘₐₓ) and area under the curve |
| nemtabrutinib | (AUC) |

### Part 1 (Dose Escalation and Confirmation) Design

Part 1 includes an open-label, non-randomized, dose escalation and confirmation design, with the aim to confirm the optimal dose of nemtabrutinib to be used in combination with venetoclax. Up to approximately 30 participants, 15 participants per dose level, will be enrolled in Part 1. Two predetermined dose levels of nemtabrutinib will be evaluated in combination with venetoclax: Dose 1: 45 mg (the starting dose level)

Dose 2: 65 mg

The end of the Dose-Limiting Toxicity (DLT) evaluation window will correspond to approximately 12 weeks after the participant has received the first dose of nemtabrutinib.

### Part 2 Design

Part 2 is a randomized, active-controlled, parallel-group, multisite, open-label study of nemtabrutinib plus venetoclax in participants with relapsed/refractory CLL/SLL.

Approximately 690 eligible participants will be enrolled and randomized to 1 of 2 groups: 15 Treatment Group: Nemtabrutinib+ venetoclax Comparator Group: Venetoclax plus rituximab (VR) (or rituximab biosimilar

In the treatment group, administration of nemtabrutinib continues for up to 2 years. Administration of venetoclax increases over 4 weeks until a dose of 400 mg is reached. In the comparator group, administration of venetoclax increases over 4 weeks until a dose of 400 mg is reached. Rituximab administration commences at 375 mg/m² and increases to 500 mg/m² for subsequent doses.

PFS is the primary endpoint in Part 2 and is defined as the time from randomization to the first documented disease progression or death due to any cause, whichever occurs first as assessed per 2018 iwCLL criteria by BICR (blinded-independent central review).

Secondary endpoints include undetectable MRD in bone marrow at Month 14 as assessed by central laboratory, OS, OR, DOR, and safety. Exploratory efficacy endpoints will include OR with response category including PRL.

Study treatment will be administered for 2 years or until unacceptable toxicity, verified disease progression (imaging or non-imaging progression), or another discontinuation criterion is met.

Part 2 will assess whether:
a. Nemtabrutinib+ venetoclax is superior to VR with respect to PFS per 2018 iwCLL criteria as assessed by BICR.
b. Nemtabrutinib+ venetoclax is superior to VR with respect to undetectable MRD rate in bone marrow at month 14 as assessed by central laboratory
c. Nemtabrutinib+ venetoclax is superior to VR with respect to OS
d. Nemtabrutinib+ venetoclax is superior to VR with respect to ORR per 2018 iwCLL criteria as assessed by BICR

### Scientific Rationale for Study Design

This is a randomized, active-controlled, parallel-group, open-label, Phase 3 study designed to evaluate nemtabrutinib, a reversible non-covalent ATP competitive inhibitor of BTK, plus venetoclax, compared to VR in participants with CLL/SLL who are relapsed or refractory to at least 1 prior therapy.

### Treatment

Treatment Group (Nemtabrutinib+ Venetoclax; Part I and Part 2) Venetoclax is administered as a daily 20 to 400 mg dose during the 5-week ramp-up phase, and then at 400 mg per day following the ramp-up phase. Venetoclax dosing continues until confirmed disease progression, unacceptable toxicity, discontinuation criteria are met, or 2 years. Nemtabrutinib is administered as a daily dose of 45 mg or 65 mg taken orally, at least 10 hours after venetoclax administration. Each cycle consists of 4 weeks (28 days).

### Comparator Group (Venetoclax+ Rituximab; Part 2)

Venetoclax is administered as a daily 20 to 400 mg dose during the 5-week ramp-up phase, and then at 400 mg per day following the ramp-up phase. Venetoclax dosing continues until confirmed disease progression, unacceptable toxicity, discontinuation criteria are met, or 2 years. Rituximab (or rituximab biosimilar is administered as an IV infusion. The initial dose is 375 mg/m², followed by 500 mg/m² for subsequent infusions.

### Timing of Dose Administration

### Venetoclax

Participants will be instructed to take the tablets QD in the morning by mouth with food and water. Venetoclax dosing escalates over a 5-week period.

### Nemtabrutinib

Participants enrolled into Part 1 or randomized to the Treatment Group in Part 2 will be instructed to take the tablets QD by mouth under fasting conditions (either 1 hour before, or 2 hours after the meal), preferably at the same time each day. Nemtabrutinib will be administered at least 10 hours after venetoclax administration.

Participants will be instructed to take the assigned dose of nemtabrutinib unless the dose has been reduced due to management of an AE. Treatment will be continued until unacceptable toxicity, documented disease progression, or another discontinuation criterion is met.

### Rituximab

Rituximab (or rituximab biosimilar) will be administered in the clinic.

First infusion: Initiate infusion at a rate of 50 mg/hour. In the absence of infusion toxicity, increase infusion rate by 50 mg/hour increments every 30 minutes, to a maximum of 400 mg/hour.

Subsequent infusions (standard infusion): initiate infusion at a rate of 100 mg/hour. In the absence of infusion toxicity, increase rate by 100 mg/hour increments at 30-minute intervals, to a maximum of 400 mg/hour.

Table I below shows details of the arms/intervention groups of the study:

TABLE 1

| | Intervention | | | | | Regimen/ | |
|---|---|---|---|---|---|---|---|
| | Group | | Dosage | Dose | Route of | Treatment | |
| | Name | Drug | Strength | Frequency | Administration | Period | Use |
| Intervention | Treatment | Nemtabrutinib | 45 and | QD | Oral | Daily, at | Test |
| Groups | Group | | 65 mg | | | least 10 | product |
| | | | | | | hours after | |
| | | | | | | venetoclax | |
| | | | | | | administration, | |
| | | | | | | until | |
| | | | | | | progressive | |
| | | | | | | disease, | |
| | | | | | | unacceptable | |
| | | | | | | toxicity, or | |
| | | | | | | discontinuation | |
| | | | | | | criteria | |
| | | | | | | met, or 2 | |
| | | | | | | years post-C2D1 | |
| | | Venetoclax (in | 20 to | QD | Oral | Daily | Test |
| | | combination | 400 mg | | | morning | Product |
| | | with | ramp- | | | dosing, until | |
| | | nemtabrutinib) | up over | | | progressive | |
| | | | 4 weeks | | | disease, | |
| | | | | | | unacceptable | |
| | | | | | | toxicity, | |
| | | | | | | discontinuation | |
| | | | | | | criteria | |
| | | | | | | met, or 2 | |
| | | | | | | years post-C2D1 | |
| | Comparator | Venetoclax (in | 20 to | QD | Oral | Daily | Comparator |
| | Group | combination | 400 mg | | | morning | |
| | | with rituximab) | ramp- | | | dosing, until | |
| | | | up over | | | progressive | |
| | | | 4 weeks | | | disease, | |
| | | | | | | unacceptable | |
| | | | | | | toxicity, | |
| | | | | | | discontinuation | |
| | | | | | | criteria | |
| | | | | | | met, or 2 | |
| | | | | | | years post-C1D1 | |
| | | Rituximab^{a} (in | 375 mg/m² | Day 1 of | IV | Once per 28- | Comparator |
| | | combination | C2; 500 | each | infusion | day cycle, for | |
| | | with | mg/m², | cycle | | a total of 6 | |
| | | venetoclax) | C3-C7 | | | cycles | |
| Total | 2 | | | | | | |
| Number of | | | | | | | |
| Intervention | | | | | | | |
| Groups/Arms | | | | | | | |

| | |
|---|---|
| Duration of | Each participant will participate in the study from the time the participant provides |
| Participation | documented informed consent through the final contact. After a screening phase of up |
| | to 42 days, each participant will receive assigned intervention. After the end of |
| | treatment, each participant will be followed for 30 days for safety. Participants will |
| | then enter either efficacy follow-up (for those who withdraw from treatment without |
| | disease progression and have not received new anticancer therapy) or survival |
| | follow-up. |

Abbreviations: C = cycle; D = day; IV = intravenous; QD = quaque die (daily).
^{a}Participants will receive either rituximab or rituximab biosimilar.

Those skilled in the art will recognize, or be able to ascertain, using no more than routine experimentation, numerous equivalents to the specific embodiments described specifically herein. Such equivalents are intended to be encompassed in the scope of the following claims.

1. A pharmaceutical composition comprising a therapeutically effective amount of a compound of Formula (I): 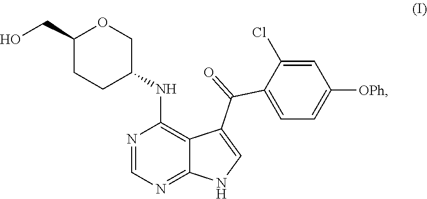 [SMILES: O=C(c1ccc(Oc2ccccc2)cc1Cl)c1c[nH]c2ncnc(N[C@@H]3CC[C@@H](CO)OC3)c12]
or a pharmaceutically acceptable salt thereof in combination with a therapeutically effective amount of at least one second agent or a pharmaceutically acceptable salt thereof, for use in the treatment of a BTK-mediated disease or disorder.

2. The pharmaceutical composition of claim 1, wherein the second agent is selected from Venetoclax, BI-97C1, sabutoclax, navitoclax, obatoclax, 4-[4-[[2-(4-Chlorophenyl)phenyl]methyl]piperazin-1-yl]-N-[4-[[(2R)-4-(dimethylamino)-1-phenylsulfanylbutan-2-yl]amino]-3-nitrophenyl]sulfonylbenzamide (ABT-737), N-[4-(2-tert-butylphenyl)sulfonylphenyl]-2,3,4-trihydroxy-5-[(2-propan-2-ylphenyl)methyl]benzamide (TW-37), APG-1252, APG-2575 or S55746.

3. The pharmaceutical composition of claim 2, wherein the second agent is venetoclax.

4. A method of treating a BTK-mediated disorder in a subject in need thereof, the method comprising co-administering to a subject in need thereof, a therapeutically effective amount of a compound of Formula (I): 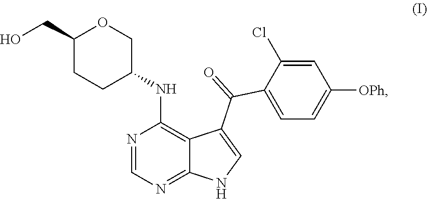 [SMILES: O=C(c1ccc(Oc2ccccc2)cc1Cl)c1c[nH]c2ncnc(N[C@@H]3CC[C@@H](CO)OC3)c12]
or a pharmaceutically acceptable salt thereof, in combination with a therapeutically effective amount of at least one second agent or a pharmaceutically acceptable salt thereof.

5. The method of claim 4 wherein a therapeutically effective amount of the compound of Formula (I), or a pharmaceutically acceptable salt thereof, is administered to a patient in need after administering a therapeutically effective amount of at least one second agent or a pharmaceutically acceptable salt thereof.

6. The method of treating a BTK-mediated disorder of claim 4, wherein the BTK-mediated disorder is a cancer selected from CLL, SLL or MLL.

7. The method of claim 6, wherein at least one second agent is selected from the group consisting of a BCL-2 inhibitor selected from Venetoclax, BI-97C1, sabutoclax, navitoclax, obatoclax, 4-[4-[[2-(4-Chlorophenyl)phenyl]methyl]piperazin-1-yl]-N-[4-[[(2R)-4-(dimethylamino)-1-phenylsulfanylbutan-2-yl]amino]-3-nitrophenyl]sulfonylbenzamide (ABT-737), N-[4-(2-tert-butylphenyl)sulfonylphenyl]-2,3,4-trihydroxy-5-[(2-propan-2-ylphenyl)methyl]benzamide (TW-37), APG-1252, APG-2575 or S55746.

8. The method of claim 7, wherein at least one second agent is venetoclax.

9. The method of claim 7, wherein about 30 to about 80 mg of the compound of Formula (I) is administered.

10. The method of claim 9 wherein about 45 mg of the compound of Formula (I) is administered.

11. The method of claim 9 wherein about 65 mg of the compound of Formula (I) is administered.

12. The method of claim 9 wherein about 80 mg of the compound of Formula (I) is administered.

13. The method of claim 8, wherein about 20 to about 400 mg of venetoclax is administered.

14. The method of claim 8, wherein about 20 to about 400 mg of venetoclax is administered.

15. A method of treating cancer comprising co-administering to a subject in need thereof a therapeutically effective amount of a compound of Formula (I): 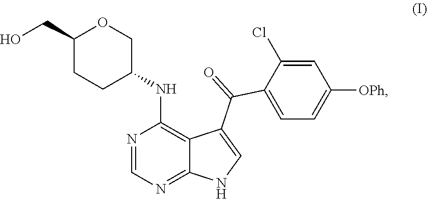 [SMILES: O=C(c1ccc(Oc2ccccc2)cc1Cl)c1c[nH]c2ncnc(N[C@@H]3CC[C@@H](CO)OC3)c12]
or a pharmaceutically acceptable salt thereof, in combination with a therapeutically effective amount of venetoclax or a pharmaceutically acceptable salt thereof, wherein the cancer is selected from CLL, SLL or MLL, and about 30 mg to about 80 mg of the compound of Formula (I) or a pharmaceutically acceptable salt thereof, is administered after administering about 20 to about 400 mg of venetoclax or a pharmaceutically acceptable salt thereof.

16. The method of claim 15, wherein 65 mg of the compound of Formula (I) or a pharmaceutically acceptable salt is administered.

17. The method of claim 15, wherein 400 mg of venetoclax or a pharmaceutically acceptable salt is administered.
